(19) United States
(12) Patent Application Publication
(10) Pub. No.: US 2010/0280135 A1
(43) Pub. Date: Nov. 4, 2010

(54) Hydrocarbon and alcohol fuels from variable, renewable energy at very high efficiency

(75) Inventor: F. David Doty, Columbia, SC (US)

Correspondence Address: Oppedahl Patent Law Firm LLC, P O Box 5940, Dillon, CO, 80435-5940, US

(73) Assignee: DOTY SCIENTIFIC, INC., Columbia, SC (US)

(21) Appl. No.: 12/673,965

(22) PCT Filed: Mar. 19, 2008
(86) PCT No.: PCT/US2008/057386

§ 371 Date: Feb. 17, 2010

Related U.S. Application Data
(60) Provisional application No. 60/895,717, filed on Mar. 19, 2007.
(60) Provisional application No. 60/896,969, filed on Mar. 26, 2007.
(60) Provisional application No. 60/945,233, filed on Jun. 20, 2007.
(60) Provisional application No. 60/969,432, filed on Aug. 31, 2007.
(60) Provisional application No. 60/977,616, filed on Oct. 4, 2007.
(60) Provisional application No. 61/017,090, filed on Dec. 27, 2007.
(60) Provisional application No. 61/026,711, filed on Feb. 6, 2008.

(51) Int. Cl. C01B 31/18; C07C 27/06; B01J 8/06

(52) U.S. Cl. 518/703; 423/418.2; 422/197

(57) ABSTRACT

A Renewable Fischer Tropsch Synthesis (RFTS) process produces hydrocarbons and alcohol fuels from wind energy, waste CO2 and water. The process includes (A) electrolyzing water to generate hydrogen and oxygen, (B) generating syngas in a reverse water gas shift (RWGS) reactor, (C) driving the RWGS reaction to the right by condensing water from the RWGS products and separating CO using a CuAlCl4-aromatic complexing method, (D) using a compressor with variable stator nozzles, (E) carrying out the FTS reactions in a high-temperature multi-tubular reactor, (F) separating the FTS products using high-pressure fractional condensation, (G) separating CO2 from product streams for recycling through the RWGS reactor, and (H) using control methods to maintain temperatures of the reactors, electrolyzer, and condensers at optima that are functions of the flow rate. The RFTS process may also include heat engines, a refrigeration cycle utilizing compressed oxygen, and a dual-source organic Rankine cycle.

### FIELD OF THE INVENTION

The field of this invention is a variable-rate Renewable Fischer-Tropsch Synthesis (RFTS) process efficiently using renewable power to produce liquid fuels from waste CO₂ and electrolyzed water by utilizing the reverse water gas shift (RWGS) reaction at moderate pressure, using effective RWGS recuperators, and recycling the unreacted FTS carbon monoxide and hydrogen at high pressure.

### BACKGROUND OF THE INVENTION

The global annual release of fossil carbon (as C) is currently over 7 billion tons, of which the U.S. contribution exceeds 20%. Currently in the U.S., 43% is from oil, 34% is from coal, and 20% is from natural gas. A comprehensive approach is needed, and it is essential for the market to help drive the dramatic cut needed in CO₂ emissions to prevent a climate disaster in this century.

There are evolving solutions. The economics for producing clean liquid hydrocarbon fuels and petrochemicals from water and waste CO₂ on wind farms improved by a full factor of eight between 2002 and 2008, seemingly without notice. Herein, we present the scientific and technological basis for another factor-of-two improvement in these economics in the next few years. Based on early-2008 commodity market trends, it will be possible within a few years to produce carbon-neutral ethanol, propanol, butanol, ethylene, propylene, methanol, ethylbenzene, cyclohexane, and hydrogen in volumes that cannot be matched by any other renewable avenue from wind energy, waste CO₂, and water. Profitable production (from wind energy) of renewable naphtha, jet fuel, diesel, gasoline, butene, and ammonia seems likely in 4 to 8 years.

Wind energy is by far our most competitive renewable energy resource, and the trends of the past decade indicate it will continue to be so for quite some time. There is enough wind energy (in class 4 sites and higher, mean wind speed above 7.2 m/s, or 16 m.p.h.) to supply seven times the world's current total electrical energy usage. Solar photovoltaic (PV) is currently about six times more expensive (per kWhr) than wind in favorable areas, and the installed cost of solar PV has increased in recent years. The perceived challenge is getting wind energy from good sites to where and when it is needed, both for the transportation sector and for the power grid. Efficient conversion of wind energy into ultra-clean, stable, liquid fuels—also called WindFuels™—solves these problems.

The concept overview shown in FIG. 1 is briefly described here to help to put the various parts of the novel fuel system into perspective. The renewable energy source would most likely be a wind farm 101. Clean water 102 and variable-rate electrical power are fed into an electrolyzer 103, which produces the hydrogen needed in the novel reverse water gas shift (RWGS) RFTS plant 104. Waste CO₂ 105—probably from a power plant—is piped into the plant 104 as well, where it and the hydrogen are converted into liquid hydrocarbon fuels, mid-alcohols, and chemical feed stocks. These fuels may then be easily stored 106 and distributed in the open market by conventional means, including pipelines 107 and tanker trucks 108. The electrolyzer also produces an enormous amount of oxygen, which may be sold if market conditions warrant, or it may be utilized in a novel refrigeration cycle or heat engine to improve the efficiency of the Renewable Fischer-Tropsch Synthesis (RFTS) plant. The water produced in the RFTS plant may be recycled through the electrolyzer. Numerous methods are disclosed for improving the efficiency of the RWGS RFTS plant.

The cost of producing chemicals and fuels from an RFTS plant will depend mostly on the quality of the wind site and on the market for the co-produced liquid oxygen. In a class-5 wind site (mean wind speed ˜8 m/s), ethanol should be profitable at $1.40/gal as long as the oxygen market is strong. In a class-4 site with no oxygen market, the cost of wind-ethanol should be about $2.70/gal. Annual wind energy production per land area in good wind regions can exceed biomass energy production density in fertile farming areas by more than a factor of five.

Fisher-Tropsch Synthesis (FTS) thermochemical gas-to-liquids (GTL) processes are widely used for producing many hydrocarbons (from CO and H₂, derived from natural gas or coal) in industrial-scale plants. (Nice summaries of the process are given by Zhang et al in U.S. Pat. No. 7,001,927, by Lowe et al in U.S. Pat. No. 7,166,643, and by AP Steynberg et al in 2007/0142481) As energy has become much more valuable over the past decade, the plant efficiencies for producing GTL diesel from natural gas have increased from about 60% to over 65%.

Methanol GTL production is simpler and more efficient, so some, including chemistry Nobel laureate George Olah, have advocated a “Methanol Economy™”. However, methanol is not a good fuel for public use in transportation: it has 5 times the toxicity and vapor pressure than was seen in the unleaded gasoline of the 1980's; a lower flash point (11° C.); and higher corrosiveness in engines. Given that public pressure has dramatically reduced the toxicity and vapor pressure of gasoline over the past two decades, the public will not accept a new motor fuel that is worse than the gasoline of the 1970's, even if there is a minor cost advantage. Renewable, carbon-neutral products that can compete in the current global market are essential to address the serious global warming challenge facing the planet today, and it appears renewable methanol will have a difficult time competing with fossil-derived methanol for the next two decades.

GTL efficiencies for more environmentally attractive fuels, such as ethanol and propanol, have usually been under 40%. In FTS literature, “higher alcohols” has generally meant “all alcohols other than methanol”, while in most other usage it usually refers to C4 and higher alcohols. Hence, we denote C₂-C₄ alcohols as “mid-alcohols” for improved clarity, especially because a significant fraction of prior “higher alcohols synthesis (HAS)” has focused on butanols, which is not the focus here. We disclose in this invention how one can obtain efficiencies above 72% for RFTS production of mid-alcohols and other products from H₂ and recovered (waste) CO₂. There will be no shortage of waste CO₂ from power plants for at least the next 50 years, and converting that CO₂ to fuels displaces the use of fossil fuels. It should be practical to extract the needed CO₂ directly from the air at reasonable cost (under $100/ton) before there is a shortage from fossil power plants.

RFTS fuels can provide the vehicle to allow wind energy to continue its phenomenal growth rate by solving the storage, intermittency, and distribution problems many have worried about with respect to alternative energy. RFTS fuels are far more easily scalable than biofuels and can ramp up as quickly as wind energy growth permits. Wind's growth rate is currently beginning to be limited by transmission-grid capacity, but RFTS completely eliminates that problem—as well as the enormous distribution and end-use costs associated with a “hydrogen economy”. If the 28% annual growth rate of wind energy of the past 14 years is maintained for another decade, wind could be providing 5% of our transportation fuel and 5% of our electrical energy needs in 2017—and its growth would halt the building of oil, gas, coal, and nuclear power plants.

The mid-sized RFTS plant described herein in some detail is small by GTL plant standards (about one-tenth the size of most current methane GTL plants), but it is still three times larger than the largest cellulosic ethanol plant currently being planned for construction by 2011. Widely noted problems with biofuels are the lack of available land to adequately handle the global oil demand and the severe effect on food prices. It is particularly noteworthy that prices of the major agricultural commodities (wheat, soybeans, corn, and oats) rose by 60% annually in 2006 and 2007, and a more recent spike dwarfed those increases. Optimistic projections indicate that even devoting all the world's arable land to biofuels production (a most untenable situation) would be insufficient to meet the world's projected demand for liquid fuels by 2030.

For more than a decade the DOE has been supporting the development of advanced nuclear power plant concepts and other concepts for the production of hydrogen. One notion in the background was that some of that hydrogen and waste CO₂ would be converted into liquid fuels (methanol, hydrocarbons, and mid-alcohols) via modified FTS plants in which the needed CO is produced by the high-temperature (HT) endothermic RWGS reaction. This idea has been picked up by others too: including Hardy and Coffey in U.S. patent publication 2005/0232833; Severinsky, in U.S. patent publication 2006/0211777; and Seymour, in U.S. patent publication 2007/0142481. But the assumption generally has been that the source of the hydrogen would be from nuclear breeder reactors (though mention has been made of renewable energy sources) and that it would be cheap, so little thought has been given to dealing with the variability issue or the details of maximizing process efficiency. As the price of uranium has increased by an order of magnitude over the past seven years and fully functional breeder reactor cycles are not expected to be available for at least 20 years, the assumption of cheap, abundant, nuclear energy seems ill founded.

The example wind-driven RFTS plant size chosen herein for illustration assumes 250 MW mean input electrical power, and it achieves 72% FTS-plant higher heating value (HHV) efficiency, or about 60% net HHV efficiency when including the electrolyzer at near-term performance. Rough analyses suggest the novel RFTS plant could be scaled down to the MW mean level and still exceed 70% efficiency at mean power for a construction cost per MW about twice that of the 250 MW plant.

The CO and H₂ conversion yields in low-temperature (LT) FTS of alkanes and alkenes have advanced to the point that the efficiency of recycling the unreacted reactants in these plants is often of minor consequence to total plant efficiency. In contrast, the need for very efficient recycling of the large amounts of H₂, CO, and CO₂ in the products from mid-alcohols FTS has possibly been the strongest argument against mid-alcohols FTS compared to gasoline, lubricants, light olefins, and diesel FTS. This argument pales in comparison to the end-use efficiency advantage mid-alcohols have over standard kerosene or diesel in ultimate engines. This is a result of the higher octane and higher autoignition temperature for mid alcohols (636 K autoignition for ethanol compared to diesel's 470 K), as these influence theoretical efficiency limits in both Otto and compression-ignition cycles. However, FTS kerosene can be efficiently highly isomerized (as in type-III aviation jet fuel) to increase its autoignition temperature even beyond that of mid alcohols, and this may permit even higher ultimate engine efficiency along with other benefits.

It is useful to note that the annual U.S. demand for the various chemicals that are not major fuel components that would come from the RFTS reactors (free of sulfur, salts, metals, halides, and nitrogen) is nearly 100 million tons, and this market is about 100 billion dollars.

Finally, it is important to appreciate that the most important factor influencing the novel RFTS plant design optimization is the rapidly changing costs in different forms of energy. In 2002, for example, the average cost of electrical energy to the U.S. industrial user was about $15/GJ, while the cost of bulk gasoline was about $6/GJ. It appears likely that the average cost of grid-quality electrical energy on wind farms in favorable regions in 2012 will be about $12/GJ (a little below its current cost), while the mean cost of bulk gasoline will probably be well over $26/GJ ($3/gal) even without a carbon tax, though the mean well-head price of natural gas will probably still be below $12/GJ in most markets. For comparison, the cost of solar PV will likely be over $50/GJ.

There seems little doubt that the price of petroleum will continue to increase at a mean annual rate of ˜15% above inflation for the next 15 years, as it will take that long for the various realistic alternatives (RFTS, wind, wave, solar, and improved efficiency) to have sufficient mitigating contributions. It seems unlikely that coal-to-liquids FTS will have a significant effect on oil prices for at least 15 years. The enormous amount of co-produced CO₂ it generates must be sequestered, and the other wastes must also be dealt with. A coal-to-diesel FTS plant typically produces only ˜0.3 kg of liquid fuels along with ˜2.2 kg of CO₂ per kg of coal, though coal-to-methanol is better. With sequestration of this CO₂, global coal reserves are sufficient to sustain global energy demand (assuming 1.5% annual growth) for about 50 years—not the oft-cited 250 years. The recent spike in the price of Asian and Australian coal to six times its price of 2003 (to ˜$100/ton, or ˜$6/GJ) should serve as a wake-up call that coal cannot be relied upon as a source of cheap energy for many years.

Some Basic Hydrocarbon and GTL Chemistry. Carbon monoxide and hydrogen readily react (exothermically) on the surfaces of appropriate catalysts at high temperatures to form various products in what is generally called a Fischer-Tropsch reaction. The products include alkanes (or paraffins, CₙH₂ₙ₊₂, the major component of gasoline and diesel), alkenes (or olefins, CₙH₂ₙ, a lesser component of fuels and a feedstock for many chemical processes), alcohols (CₙH₂ₙ₊₁OH), methane, and many others. One major chemical engineering task is to come up with catalysts and conditions (H₂/CO/CO₂ ratio, temperature, pressure, and gas velocity) that yield as high a fraction as possible of the desired hydrocarbons.

Ethanol and octane, for example, are produced according to the following exothermic reactions. Note that all reaction heats herein are given at 600 K, as that is the FTS-relevant temperature, so values are a little different from the more commonly seen numbers.

2CO+4H₂→C₂H₅OH+H₂O, ΔH=−272kJ/mol(3:1 vol. reduction), [1]

8CO+17H₂→C₈H₁₈+8H₂O, ΔH=−1282kJ/mol [2]

However, the FTS product is always a mixture of many different hydrocarbons; and various separations and upgrading operations are needed to achieve adequate purity and to efficiently convert the less valuable hydrocarbons into desired products.

The GTL plant usually has four major components: syngas generation, syngas purification, FTS, and product upgrading. In most GTL plants, the syngas comes from natural gas, mostly via the following endothermic reforming reaction:

CH₄+H₂O→CO+3H₂, ΔH=218kJ/mol(1:2 volume increase), [3]

and the exothermic water gas shift (WGS),

CO+H₂O [symbol] CO₂+H₂, ΔH=−38.9kJ/mol, [4]

which, as indicated by the above notation, is reversible, though perhaps not very close to equilibrium at the reactor outlets. Additional CO may be generated to get the desired H₂/CO ratio by exothermic partial oxidation,

2CH₄+O₂ [symbol] 2CO+4H₂, ΔH=−54.2kJ/mol, [5]

or by endothermic CO₂ reforming,

CH₄+CO₂ [symbol] 2CO+2H₂, ΔH=257kJ/mol. [6]

In natural gas (NG) GTL plants, and even more so in biomass or coal GTL plants, a huge amount of effort and cost must be put into syngas control and clean up, as contaminants can quickly deactivate the FTS catalysts. (Sulfur, the most critical, needs to be well below 0.5 ppm, preferably below 0.05 ppm; but NH₃, tars, NORₓ, halides, metals, salts, and HCN must also be extremely low.) In a typical NG GTL methanol plant, the syngas production section amounts to over half the capital cost of the plant—and usually over 70% of the cost in biomass GTL methanol plants. In the novel RFTS plant, the hydrogen is generated at very high purity (over 99.95% after drying) from water electrolysis. Assuming the waste CO₂ is well cleaned (which is easy to do) before it is used to generate the needed CO (by the reverse of equation [4]), the hot syngas cleanup problem is avoided, and this allows cost savings in the plant as well as reduced efficiency losses.

If the exothermic FTS reactions could be carried out at higher temperatures than the endothermic production of the syngas, the latter could be driven by a fraction of the heat from the exothermic reactions. This is thermodynamically impractical for methane reforming, but when the starting reactants are H₂ and CO₂, this may be practical.

While the hydrocarbon synthesis from CO₂ and H₂ always occurs in at least two steps, as seen above, the stoichiometry of the overall reaction may be represented by a single useful equation. For example, ethanol would be:

2CO₂+6H₂→C₂H₅OH+3H₂O+energy [7]

From mass balance and the heats of combustion of hydrogen and ethanol, one readily calculates that the theoretical maximum chemical efficiency of this synthesis is 80.1%. (For perfect conversion, 26 kg of H₂ (3690 MJ) plus 192 kg of CO₂ yields 100 kg of C₂H₅OH (2970 MJ) plus 117 kg H₂O, plus 720 MJ waste heat. One obtains the 26 kg of H₂ needed by electrolyzing 234 kg of H₂O, and in the process also generates 208 kg of waste O₂.) If the excess heat is released from the FTS reactor at, for example, 620 K, then a heat engine between this source and 310 K could theoretically recover up to half of the heat of the reaction, suggesting a theoretical combined-cycle limit of 90% efficiency. For diesel or gasoline from either methane or H₂+CO₂ the theoretical chemical efficiency limit is about 77%, or about 85% if an ideal heat engine is added. A recent GTL-diesel plant has reported over 65%. One advantage of ethanol may be appreciated by noting that its synthesis from H₂+CO₂ results in 1.5 molecules of water per carbon atom in the fuel, while the synthesis of alkanes or alkenes results in 2 molecules of water per carbon atom in the fuel.—

Deficiencies of Prior FTS Plants. Prior GTL ethanol efficiencies have usually been below 40%. There are at least ten reasons, beginning with the smallest first:
1. Prior mid-alcohols GTL synthesis has been from methane, not hydrogen, and the theoretical chemical efficiency limit there is 3-11% lower (depending on what one assumes about the energy source for the syngas production).
2. A mixed-alcohols plant must operate at 50-120 bar, compared to 15-40 bar for diesel or gasoline FTS. The losses associated with the required compressors and expander turbines have often amounted to more than 6%, partly because there has been inadequate concern about non-isentropic expansions of FTS product gases. With recent advances in the technology and the benefits of mass production, the equipment needed to keep these losses under 3%, even in a small (50 MW) RFTS plant, becomes fairly inexpensive.
3. Big efficiency losses in small plants have occurred in wasted byproducts, the post-FTS product separations, and upgrading. Except in the production of methane or methanol, many hydrocarbons other than the ones specifically desired are always formed along with the preferred species. For an H₂+CO₂ source, most of these have theoretical chemical efficiency limits between 75% and 83%, so their direct effect on total chemical conversion efficiency is small if they can be efficiently utilized. Byproduct upgrading will be much easier in the RFTS plant, as shown later.—
4. Prior catalyst development has usually been constrained by the need for good tolerance of sulfur (and other poisons) and for the need for high CO conversion per pass, neither of which is needed in the novel RFTS plant.
5. Enormous effort and cost must be put into cleanup of the syngas from any fossil or biomass source.
6. Substantial efficiency losses have been associated with the required gas separations (CO₂, H₂O, CO, CH₄, H₂, light HCs, N₂, O₂, etc.). Most mixed-alcohols demo plants have borrowed product separations processes that were developed and optimized in the early 1970's in different industries (petroleum, fermentation, homogeneous catalysis, etc.) for conditions radically different from a high-pressure FTS product stream with high gas fractions.
7. As there has been almost no commercial experience in GTL of mid-alcohols, many of the demos have operated at conditions (H₂/CO ratios, pressures, and temperatures) more appropriate for alkanes and alkenes than for mid-alcohols, partly because of equipment limitations (especially compressors and expanders).
8. Usually the very light hydrocarbons and much of the unreacted syngas have been sent to a gas turbine (often of only 30% efficiency) for power generation rather than upgrading or recycling. There has been substantial progress in separation technologies (cryogenic methods, adsorbents, and membranes) over the past three decades.
9. The enormous amount of waste heat generated in the FTS reactor has not previously been very efficiently utilized. A novel Dual-source Organic Rankine Cycle, the subject of a separate pending patent application, will allow this mid-grade waste heat to be converted to electrical power at 50% efficiency when sufficient amounts of low-grade waste heat are also available—as is the case when hydrogen is being produced by electrolysis.
10. Often there has been little value ascribed to the H₂ byproduct generated in the FTS reactor from the water gas shift (WGS), which has sometimes amounted to 80% of the total loss. Now, in the H₂/CO₂-fed plant, the H₂ and CO₂ from the WGS are indistinguishable from the initial reagents and may be more readily recycled.

Most of the points listed above also apply to all small, experimental HT-FTS plants, and some of them apply to existing light-olefins HT-FTS plants, where there has been limited experience. These points are addressed in the novel RFTS plant design, and other innovations are also presented, including some future possibilities. For example, the endothermic syngas generation has previously always been carried out far above the FTS reaction temperature—usually at 1100 K or higher, and at 1.5-3 MPa (15-30 bar). Hence, the syngas generation has had to be driven by an additional heat source. For the H₂+CO₂.fed RFTS plant, it may be possible to drive the endothermic syngas reaction with waste heat from the exothermic FTS reaction if the FTS reaction temperature can be increased sufficiently. However, even if this is not yet practical, the heat needed now for endothermic CO production is much less than for methane reforming.

GTL Catalysts. Low-temperature FTS reactors (450-540 K) have some advantages: much better selectivity for diesel production, reduced reactor construction costs, much less methane production, and less coking and sintering of the catalysts. However, some FTS reactors have operated at high temperatures (540-710 K) for improved selectivity of light olefins, gasoline, and mid-alcohols. There is now more motivation for doing the FTS at the highest practical temperature: (a) the products that are more highly selected at higher temperatures have become more valuable, (b) the FTS waste heat can be more efficiently converted to electricity, and (c) it may become possible to drive the endothermic syngas production from H₂ and CO₂ directly using the FTS heat.

Sulfide catalysts, mainly MoS₂ with some CoO, have recently been used for high-temperature production of mixed-alcohols (570-630 K, 3-18 MPa) because selectivity to mid- and higher alcohols can be up to 90%. These require rather low CO₂ in the syngas, and the sulfide catalysts do not last with low-sulfur syngas. A high-sulfur syngas is clearly unacceptable, as it will poison all the other catalysts in the plant and require expensive clean up of the products. Also, the sulfide catalysts tend to produce more CH₄.

Some of the best early results for high-temperature production of mixed-alcohols were obtained with a modified Fischer-Tropsch process, with some CO₂ in the syngas, using alkali/CuO/CoO catalysts at 550-630 K, 6-20 MPa. These catalysts are quite sensitive to sulfur, though that is not an issue in wind-fuels. With the low H₂/CO ratio needed for mid-alcohols, the liquid product prior to separation typically contains 30-50% mid-alcohols. Promising results have also been obtained with K—Co—Mo/Al₂O₃ at 620 K, 10 MPa. Adequate performance with Mo-based catalysts has been obtained with up to 30% (molar fraction) CO₂ in the syngas. Interesting mid-alcohols results have recently been reported for Cu/ZrO₂ for lengthy runs (up to 2000 hours) at 570-650 K, 9-12 MPa, and for K/Zr/Zn/Mn at up to 700 K and up to 25 MPa. Modified methanol catalysts have also shown promise—such as alkali/ZnO/Cr₂O₃, which has worked at over 690 K. Possibilities have also been shown for carbon-coated cesium-promoted Cu/Zn-chromite and iron nitride catalysts for mid-alcohols and other higher oxygenates in high-pressure HT-FTS reactors.

In general, there is a trade-off between maximizing CO conversion and maximizing yield of mid-alcohols, which emphasizes the importance of efficient CO recycling in the mid-alcohols plant—a feature that has generally not been well implemented. By accepting quite low CO conversion (under 30%), very high selectivity to mid-alcohols with high yield has very recently been shown for K₂CO₃-promoted β-Mo₂C catalyst at 573 K, 8 MPa, H₂/CO=1.

Very high selectivity of the C₂-C₄ olefins has been demonstrated using Fe/MnO/SiO/K catalysts in HT-FTS reactors, and the selectivity to ethylene may be further improved by reducing the reactor pressure and increasing the temperature (though both such changes increase the rate of catalyst degradation from carbon deposition). Selectivity to both light olefins and mid-alcohols relative to methane has been shown to improve with increasing feed CO₂, though its mole fraction needs to be limited to about 15% and feed water must be kept very low to limit acid production. Undoubtedly, there is scope for considerable improvement in these catalysts and conditions.

The best current high-temperature technology for the production of gasoline and diesel may be Fe/K catalysts at 550-620 K, 1-4 MPa, though low-temperature FTS processes (450-540 K) are much better for maximizing diesel. The maximum gasoline fraction per pass is about 40%. Wax is a major byproduct unless the Sasol HT fluidized-bed process is used, but wax can easily be converted to high-value lubricants, diesel, etc. Catalyst lifetime can be as short as several months, even with clean reactants, but the catalyst is cheap and can be readily replaced or continuously rejuvenated in a properly designed reactor. The addition of 6-12% molybdenum to the Fe/K catalysts has been shown to improve their stability and lifetime in HT reactors.

The first slurry-bed (bubble column) reactors came on line in the 1990s and permitted substantial reactor size and cost reductions as well as improved process condition control, but they were only effective with low-temperature catalysts. More recently, they have been extended to higher temperatures and have shown some promise for mid-alcohol production. The 2-phase fluidized-bed Sasol reactor has proven superior for HT-FTS of gasoline from methane. However, an advanced-design fixed-bed reactor appears better for mid-alcohols, as will be shown.

Commercial HT-FTS plants for enhanced production of light olefins have been proposed for at least a decade, but until quite recently the market value of light olefins relative to that of lubricants and other products has not been high enough to push the product balance in this direction as far as now seems optimum. An example product mix in a previously published light-olefin HT-FTS plant was: ˜23% diesel, ˜19% propylene, 13% gasoline, 13% butenes, 12% mid-alcohols and other oxygenates (mostly acetic acid and acetone), 10% ethylene, 4% LPG, 3% ethane, and 4% other. There would now be strong profit incentive in a wind-driven light-olefin plant to reduce the diesel and gasoline in favor of more mid-alcohols and light olefins.

Methanol may be produced with 99% yield (with recycling), and that is the reason it has been the most common GTL product. It has been produced at up to 630 K and 30 MPa using ZnO/Cr₂O₃ catalysts, but recent trends are toward much lower temperatures (490-570 K) using Cu/ZnO/Al2O3 at 5-15 MPa. There will always be industrial need for methanol, but it is not a good commercial motor fuel, as noted earlier. The mid-alcohols plants will eventually produce as much methanol byproduct as needed to satisfy all industrial applications.

Efficient, RWGS, Syngas Production. As noted by many, including O'Rear, in U.S. Pat. No. 6,846,404, the endothermic reverse water gas shift reaction is given by:

CO₂+H₂ [symbol] CO+H₂O, ΔH=38.9kJ/mol. [8]

A few inventors, including Hardy and Coffey in U.S. patent publication 2005/0232833, have also noted the limitations of using hydrogen as an energy carrier and have proposed using waste CO₂ and hydrogen from electrolyzed water to produce the needed CO via the RWGS.

The reverse of the RWGS, the WGS, is easy to achieve at low-temperatures (450-550 K) and high pressures using Cu/ZnO catalysts, but the needed low temperature RWGS has seen relatively little investigation and utilization. Generating syngas from CO₂+H₂ has not been an objective of much prior work, due to the expense of H₂ from electrolyzed water compared to the cost of methane. Until now, the market has not had a well articulated need for an optimum low-temperature RWGS catalyst. The RWGS reaction has often been seen as an undesirable competing reaction to be suppressed—as in methanol synthesis.

There are several exothermic reactions competing with the RWGS:

CO₂+3H₂ [symbol] CH₃OH+H₂O, ΔH=−61.5kJ/mol, [9]

CO₂+4H₂→CH₄+2H₂O, ΔH=−179kJ/mol, [10]

The following exothermic reactions and the exothermic reverses of eqs. [3] and [6] compete when CO is present in sufficient amounts.

CO+H₂→C+H₂O, ΔH=−135kJ/mol, [11]

and 2CO→C+CO₂, ΔH=−174kJ/mol. [12]

### (And to Repeat, all Reaction Heats Herein are at 600 K.)

At the pressures and temperatures appropriate to optimize the RWGS relative to methane, methanol production is usually negligible. Moreover, in synthesis of mid-alcohols, considerable CH₃OH may simply be fed into the FTS reactor, where it may be converted to mid-alcohols. Alternatively, if there is significant methanol production and if it is not desired in the FTS reactor (as for light olefins production), it may be partially decomposed below 590 K (using FTS reactor waste heat and Cu—Zn catalysts) according to

CH₃OH [symbol] CO+2H₂, ΔH=100.4kJ/mol. [13]

Carbon deposition—leading to catalyst deactivation—is usually dominated by the Boudouard reaction, eq. [12]. Its activation energy is rather high (113 kJ/mol), but it is critical that it not be catalyzed. Of course, reducing the CO partial pressure will quickly reduce the reaction rates of eqs. [11] and [12] and the reverses of eqs. [3] and [6].

Some have thought that the RWGS cannot be made to work adequately below 720 K, and this may be true at high reactor pressures (over 5 MPa) with high space velocities and low excess CO₂ and H₂. However, it is not difficult to accommodate excess H₂ and CO₂ in the product stream, low reactor pressures, and moderate space velocity.

A catalyst with good selectivity to CO is needed to keep CH₄ down, especially at higher pressures. Some of the more effective RWGS catalysts for the 520-720 K range at 0.3 to 3 MPa (total pressure) include Au/TiO₂, Cu/silica, and Cu/alumina. Even higher selectivity (98%) and excellent activity have been reported for a 0.9%-Pt doped Ca/C catalyst at low temperatures, though it is rather expensive. With optimum space velocity, it appears that methane and carbon production can be kept low by operating below 1 MPa H₂ partial pressure at lower temperatures (below 660 K), or below 0.3 MPa H₂ partial pressure at higher temperatures—at least to 700 K with Cu catalysts, and possibly to 970 K with Fe₃O₄/Cr₂O₃ or future catalysts. Most data (some of which are reported later) thus far for plausible conditions (well above atmospheric pressure, low methane yield, low-cost catalysts, low C deposition, and high CO yield) are also at rather low space velocity, so more development here could improve performance and reduce reactor cost.

The dominant limitation in the catalyzed RWGS reaction is the WGS reaction, as the reverse is always also catalyzed. The easiest way to reduce the WGS is to condense the water from the mixture after partial reaction, and then re-heat and repeat this cycle as necessary. This can permit high conversion of the CO₂ to CO (though CO₂ separation from the product will still be needed) with little additional energy penalty if highly effective counterflow heat exchangers are employed. Suitable exchangers for low-pressure operation (which is critical for keeping methane low with metal catalysts) may not be commercially available, but they have been shown to be feasible at reasonable cost.

The RWGS reaction may be driven further to the right by including CO removal from the products as the reaction progresses. Several methods for CO separation have been demonstrated. The most widely implemented is the COSORB method of Kinetics Technology International (originally developed by Tenneco Chemicals), which uses a solution of CuAlCl₄ in toluene for the selective absorption of CO from mixtures containing CO₂, H₂, CH₄, and inerts. The Cu(I)—CO complex is formed at about 300 K and moderately high pressures (0.3-3 MPa), and the CO is released at about 400 K and low pressures (0.1-0.5 bar). Semi-permeable membranes and molecular sieves (such as zeolite 5A) are also available with fairly good selectivity for CO. All of these methods are more expensive than simple H₂O condensation, and most add significant additional gas compression penalties. However, the combination of CO and H₂O removal from the products may allow the RWGS reaction to work adequately below the FTS reaction temperature, and that benefit should eventually more than offset the costs associated with CO separations.

Initially, most of the heat needed to drive the endothermic RWGS reaction would probably come from combustion of lowest-value byproducts from the FTS reactions—primarily methane. Some of the heat may come from reforming of low-value FTS products (methane, ethane, and propane) into syngas using an exothermic partial oxidation. Concentrated solar heat could also often be used—even at night, with thermal storage.

Severinsky, in U.S. patent publication 2006/0211777, has recognized the potential value of using the FTS waste heat to drive the RWGS reaction, though this obtuse publication provides little if any of the information needed for actual reduction to practice. Severinsky also notes that theoretically it is not necessary for the RWGS temperature to be below the temperature of the FTS reactor to get a significant portion of the RWGS heat needed from the FTS reactor. In theory, a heat pump could pump heat from the FTS reactor to a higher temperature (perhaps 720 K) with much less electrical power than would be required for direct heating of RWGS reactors. However, effective heat pumps for this temperature range have not been shown to be practical; and even if possible, they would be quite expensive and achieve at best a factor of two reduction in the amount of electrical input power required.

With current catalysts the FTS production of light olefins, gasoline, or mid-alcohols can work with good selectivity, adequate lifetime, and acceptably low coking and methane production up to at least 610 K, making it easier to utilize its waste heat more efficiently. However, this is still not high enough to readily drive the RWGS reaction, at least under variable conditions—except perhaps if both the CO and the H₂O in the RWGS are held to low levels. The amount of heat required for the RWGS is at least 8% that of the total FTS products. However, the difference in system efficiency between the two options (burning low-value byproducts or using FTS heat) is only about half that amount if highly effective methods are available for conversion of waste heat to electrical power, as discussed briefly in the next section. Nonetheless, the potential efficiency advantage of driving the RWGS with FTS waste heat provides incentive to develop catalysts and plant designs compatible with higher FTS temperatures and lower RWGS temperatures.

While the WGS is not a significant loss route in LT-FTS diesel or gasoline reactors, WGS activity in HT-FTS reactors can be quite high. Previously, there has not been a very good method for utilizing the WGS products and waste heat. However, when the syngas is being generated by the RWGS, the WGS byproducts can readily be converted back into syngas.

Approaching Second-law Limits in Waste Heat Utilization. There are two huge and comparable sources of waste heat in the wind-fuels plant—the electrolyzer and the FTS reactor. Initially, these will probably be at just 10 K above current best practice in related applications (i.e., about 430 K and 610 K respectively), and each would be rejecting 30-60 MW in a 250 MW wind-fuels plant. Some of this can be used directly in preheating of reactants and in distillations of products, but most will need to be converted into electricity as efficiently as possible.

Over the past four decades, a large number of variations on the Organic Rankine Cycle (ORC) have been evaluated for the purpose of improving the economic utilization of low-grade heat, as available from geothermal sources (usually 360 K to 440 K) or mid-grade heat, as available from concentrated solar power (CSP, 480 K to 750 K). Yet, it seems that few have exceeded 55% of the second-law efficiency limits. This is largely because the latent heat of vaporization and the differences in specific heats between the liquid and gas phases make full optimization (minimizing irreversibilities) impossible for a single heat source. In addition, ORCs have generally been very expensive, partly because of poor appreciation for the importance of a high condenser pressure in minimizing exchanger costs. We show in a separate application (international patent application number PCT/U.S.07/85484, filed Nov. 25, 2007, incorporated herein by reference) how a novel Dual-source doubly-recuperated Organic Rankine Cycle (DORC) allows one to achieve efficiencies much closer to second-law limits while simultaneously reducing the cost and complexity of the heat engine when both a low-grade and a mid-grade heat source (of comparable magnitudes) are available and the working fluid is optimally selected. The novel design is related to the dual-source steam Rankine cycle disclosed by Martin et al in U.S. Pat. No. 3,950,949. Simply put, two different heat sources are much better than one.

The importance of approaching isothermal conditions in heat transfer has been understood—for more than three decades, but methods of doing so in gas-to-gas recuperators have had very limited success. Some methods of improving high-effectiveness heat transfer are discussed later in the Detailed Description, and an order of magnitude improvement in cost-effectiveness of gas-to-gas recuperators is the subject of another pioneering, co-pending patent application, namely U.S. application No. 61/034,148, filed Mar. 5, 2008 and incorporated herein by reference for all purposes.

Some Other Relevant Art. Hensman et al in U.S. Pat. No. 7,115,670 have nicely disclosed a method of improving temperature uniformity and control in an FTS reactor by combining internal heat exchangers with re-circulation of a suspension of the liquid products, catalysts, and gas bubbles through an external heat exchanger and re-injecting this slightly cooled suspension at high velocity to enhance mixing and temperature uniformity. Liquid sodium is mentioned as a possible working fluid, but sodium is a highly reactive metal that is difficult to work with and thus is expensive, even though it is very abundant. They prefer the use of rather cool water as the coolant fluid, apparently to allow the heat exchangers to be smaller, though this increases thermal gradients within the reactor and makes efficient high-grade heat recovery for other useful purposes impossible. They also prefer the use of reactors of about 8 m in diameter and 20 m length for liquid product production rates of 30,000 bbl/day, or about 50 kg/s, at pressures of about 25 bar, temperature of about 500 K, gas residence time in the reactor of about 40 s, and reactor heat removal requirement of about 550 MW. The example size presented herein is about one-tenth this size, and the reactor heat removal requirements are proportionally much less, as the reaction heats are much lower.

As noted earlier, one area usually seeing substantial losses in the GTL plant—especially in smaller plants—is in the gas compressions and expansions, as the FTS reactor needs to operate at 0.5 to 20 MPa (5 to 200 bar). While typical GTL compressor efficiencies have been under 80%, multi-stage compressors have been able to exceed 91% efficiency for at least two decades. Moreover, there has been considerable further progress in turbine and compressor optimization over the past decade. Inter-cool may be used to reduce the work input needed for high compression, though this may not be advantageous if the product then needs further heating—unless excess waste heat is available. Tranier shows the value of isentropic expansions in a cryogenic gas separations plant in U.S. Pat. No. 7,143,606. It may be interesting to note that, for nearly two decades, Doty Scientific, of Columbia S.C., USA, has been producing simple, single-stage, micro expander turbines that achieve 40% polytropic efficiency at flow rates three to four orders of magnitude smaller than needed in the 250 MW RFTS plant.

Behens has suggested in U.S. Pat. No. 7,302,903 that wind energy should be used to produce hydrocarbons from seawater in floating vessels. One of the many problems with his concept is that it utilizes CO₂ that has already been sequestered in the ocean. Seymour, in U.S. Pat. No. 7,238,728 and elsewhere, has suggested unclear processes that might achieve efficiencies an order of magnitude lower than can be achieved by the process laid out herein.

Martin and Kubic have recently suggested that a novel approach to electrolytic rejuvenation of an aqueous solution of CO₂-laden K₂CO₃ may permit low-cost separation of CO₂ from the atmosphere if enormous amounts of low-cost high-quality water and low-grade heat are also available. They then propose that this CO₂, along with hydrogen from water electrolyzed by nuclear energy, be converted to methanol in a process based on eq. [9] which they call “Green Freedom™”. They apparently favor nuclear energy because their process is not sufficiently efficient to be competitive if renewable energy is utilized, and they believe very large nuclear power plants can produce hydrogen at low cost.

Designing for Variability. A widely noted characteristic of wind and solar energy is that they are variable. Wind and solar installations are customarily rated according to their peak power capability. Mean power generation in a typical Class 5 wind site is about 35% of the peak capability of the hardware. Mean solar power is typically only 28% of peak, though its diurnal cycle is a usually a much better match to the grid demand load than is wind's. Herein, plant size refers to its mean power, as this allows for fairer comparisons to conventional power plants and perhaps will begin a trend in that direction. However, the assumption is that the RFTS plant needs to be able to operate, essentially continuously, at three times this mean rating and perhaps at one-tenth, or at least one-third, this mean rating. Wave, geothermal, hydrokinetic, and tidal energy, which seem likely to be more practical than solar PV in many areas, are much less variable; and this would allow considerable savings in an RFTS plant driven by such sources compared to one driven by wind energy.

The hydrogen production rate from the electrolyzers at the plant would be able to change as quickly as needed in response to changing wind conditions as long as the electrolyzer is maintained near optimum operating temperature and pressure. However, the RFTS plant would not be able to respond as quickly, so some local hydrogen storage would be needed—preferably at least 6 hours worth—for efficient power-down, standby, and power-up cycles. For a plant of 250 MW average power, that comes to a fairly substantial amount—about 30 tons (360,000 m³ at STP). While compact, light-weight hydrogen storage in small quantities (as needed for fuel-cell vehicles) is quite expensive, bulk hydrogen-gas storage at moderate pressure (1-15 MPa) is not—around $400,000/ton, or about $12M for the 250 MW plant (which will cost $1B total, including the wind farm). Modest carbon monoxide storage would be sufficient, as the syngas generator would respond about as fast as the Fisher-Tropsch section of the plant. However, some CO storage—perhaps 50 tons—would improve transient response time and greatly simplify control during transients, as also would the storage of some hydrogen at several different temperatures and pressures. (The storage cost for CO is an order of magnitude less than that for H₂.) Some on-site CO₂ and water storage would also be needed, even if they were being piped in from very reliable sources.

The huge amount of compressed oxygen byproduct being produced by the electrolyzers may saturate the local oxygen market, so it is useful to find a way to use some of it on site. It will be shown that cryogenic refrigerators and heat engines operating off this free compressed oxygen (at 2-15 MPa, as for the H₂) may be advantageously used in the novel RFTS plant for improved plant efficiency. Sufficient amounts of compressed or liquid oxygen (LOX) would normally be stored for efficient refrigerator operation during power down, standby, and power up. The 250 MW plant, for example, would produce nearly 40 tons per hour. Such a plant may need to store more than 200 tons of LOX. The above suggested H₂ and LOX minimum storage amounts are about one-third the fuel-up requirements for the space shuttle and are not difficult to accommodate safely. Extended hydrogen storage in quantities larger than ˜50 tons may be better handled as a cryogenic liquid. The availability of other on-site cryogenic facilities (perhaps 110 K for LOX at 0.7 MPa) and novel gas-to-gas recuperators help make this option more efficient. In this way, it would be practical to cost effectively accommodate even months of excess wind capacity followed by months of light winds.

Nonetheless, it is likely that the first demonstrations of RFTS will be in areas where there is excess grid capacity and where grid power, perhaps supplemented by renewable energy, is currently cheap. Such a site might be over 300 km from where most of the renewable energy or any other input originates, as distribution costs for all of the inputs and outputs (electricity, CO₂, water, liquid fuels, O₂, chemicals, waste heat, etc.) must be weighed. The RFTS demo plants can then be designed for constant-rate operation off the grid. Substantial challenges in RETS will arise from dealing with the major variabilities in wind and solar with limited hydrogen storage.

### RELEVANT ART

1. A P Steynberg and ME Dry, eds. Studies in Surface Science and Catalysis 152, Fischer-Tropsch Technology, Elsevier, 2004.
2. S Phillips, A Aden, J Jechura, and D Dayton, “Thermochemical Ethanol via Indirect Gasification and Mixed Alcohol Synthesis of Lignocellulosic Biomass”, NREL/TP-510-41168, 2007. http://www.nrel.gov/docs/fy07osti/41168.pdf
3. K Ibsen, “Equipment Design and Cost Estimation for Small Modular Biomass Systems. Task 9: Mixed Alcohols from Syngas—State of the Technology”, NREL/SR-5,0-39947, 2006. http://www.nrel.gov/docs/fy06osti/39947.pdf
4. P L Spath and D C Dayaton, “Preliminary Screening—Technical and Economic Assessment of Synthesis Gas to Fuels and Chemicals with Emphasis on the Potential for Biomass-Derived Syngas”, http://www.fischer-tropsch.org/DOE/DOE_reports/510/510-34929/510-34929.pdf, NREL/TP-510-34929, 2003.
5. R Zubrin, B Frankie, and T Kito, “Mars In-Situ Resource Utilization Based on the Reverse Water Gas Shift Experiments and Mission Applications”, AIAA 97-2767, 1997, http://www.marssociety.de/downloads/Artikel/in-situ.pdf
6. K Weissermel, H J Arpe, Industrial Organic Chemistry, 4th ed., Wiley, 2003.
7. M Xiang, D Li, H Qi, W Li, B Zhong, Y Sun, “Mixed alcohols synthesis from CO hydrogenation over K-promoted β-Mo₂C catalysts”, Fuel 86, 1298-1303, 2007.
8. David G Wilson and Jon Ballou, “Design and Performance of a High-Temperature Regenerator Having Very High Effectiveness, Low Leakage and Negligible Seal Wear”, paper GT 2006-90096, Turbo-Expo 2006, Barcelona.
9. J A Hogendoorn, W P M van Swaaij, G F Versteeg, “The absorption of carbon monoxide in COSORB solutions: absorption rate and capacity”, Chem. Engr. J. 59, 243-253, 1995, http://doc.utwente.nl/11240/1/Hogendoorn95absorption.pdf
10. G Olah and A Molar, “Hydrocarbon Chemistry”, 2nd ed., Wiley, 2003.
11. C H Bartholomew and R J Farrauto, Industrial Catalytic Processes, Wiley, 2006.
12. J D Seader and E J Henley, “Separation Process Principles”, 2nd ed., Wiley, 2006.
13. J Ivy, “Summary of Electrolytic Hydrogen Production”, NREL/MP-560-36734, 2004. http://www.nfpa.org/assets/files/PDF/CodesStandards/HCGNRELElectrolytichydrogenproduction04-04.pdf
14. K Schultz, L Bogart, G Besenbruch, L Brown, R Buckingham, M Campbell, B Russ and B Wong, “Hydrogen and Synthetic Hydrocarbon Fuels—a Natural Synergy”, General Atomics, 2006. http://bioage.typepad.com/greencarcongress/docs/HydrogenSynfuel.pdf
15. BR Smith, “XCELPLUS' Business Plan for Building Coal to Ethanol Plants on the Eastern Seaboard”, 2006 http://www.xcelplusglobal.com/company/business_plan.pdf
16. G P Huffman, “C1-Chemistry for the Production of Ultra-Clean Liquid Transportation Fuels and Hydrogen”, Consortium for Fossil Fuel Science, Univ. Kentucky, 2003. http://www.osti.gov/bridge/servlets/purl/881866-rjnpCh/881866.PDF
17. J I Levene, “Economic Analysis of Hydrogen Production from Wind”, WindPower 2005, Denver, NREL/CP-560-38210, 2005. http://www.nrel.gov/docs/fy05osti/38210.pdf
18. R Bourgeois, “Advanced Alkaline Electrolysis”, DE-FC36-04G014223, GE Global Research Center, 2006. http://www.hydrogen.energy.gov/pdfs/review06/pd_{—}8_bourgeois.pdf.
19. J Underwood, “Design of a CO₂ Absorption System (K₂CO₃ method) in an Ammonia Plant”, see http://www.owlnet.rice.edu/˜ceng403/co2abs.html
20. L R Rudnick, “Synthetics, Mineral Oils, and Bio-based Lubricants: Chemistry and Technology”, CRC, Boca Raton, 2006.
21. M Kanoglu, “Exergy analysis of a dual-level binary geothermal power plant”, Geothermics, 31, 709-725, 2002.
22. DESIGN II for Windows Tutorial and Samples Version 9.4, 2007, by WinSim Inc., available from http://www.lulu.com/includes/download.php?fCID=390777&fMID=810115.
23. J E Whitlow and C Parrish, “Operation, Modeling and Analysis of the Reverse Water Gas Shift Process”, 2001 NASA/ASEE summer program, JFK Space Center, http://ntrs.nasa.gov/archive/nasa/casi.ntrs.nasa.gov/20020050609_{—}2002079590.pdf
24. J F Martin and W L Kubic, “Green Freedom, A concept for Producing Carbon-Neutral Synthetic Fuels and Chemicals”, Los Alamos National Laboratory, 2007, http://www.lanl.gov/news/newsbulletin/pdf/Green_Freedom_Overview.pdf

U.S. PATENT DOCUMENTS

| | | | |
|---|---|---|---|
| 3,950,949 | April 1976 | Martin et al | 60/641 |
| 4,099,381 | July 1978 | Rappoport | 60/641 |
| 4,304,585 | December 1981 | Oda et al | 65/43 |
| 4,460,384 | July 1984 | Hirai et al | 95/178 |
| 4,676,305 | June 1987 | Doty | 165/158 |
| 5,030,783 | July 1991 | Harandi et al | 585/322 |
| 5,232,474 | August 1993 | Jain | 55/26 |
| 5,259,444 | September 1993 | Wilson | 165/8 |
| 5,609,040 | March 1997 | Billy et al | 62/622 |
| 6,178,774 | January 2001 | Billy et al | 62/620 |
| 6,277,338 | August 2001 | Agee, Weick | 422/189 |
| 6,572,680 | June 2003 | Baker et al | 95/51 |
| 6,660,889 | December 2003 | Fujimoto et al | 568/429 |
| 6,846,404 | January 2005 | O'Rear | 208/133 |
| 6,939,999 | September 2005 | Abazahian | 585/640 |
| 7,001,927 | February 2006 | Zhang et al | 518/700 |
| 7,084,180 | August 2006 | Wang et al | 518/712 |
| 7,115,670 | October 2006 | Hensman et al | 518/712 |
| 7,143,606 | December 2006 | Tranier | 62/611 |
| 7,166,219 | January 2007 | Kohler et al | 210/617 |
| 7,166,643 | January 2007 | Lowe et al | 518/700 |
| 7,227,045 | June 2007 | Ansorge et al | 568/451 |
| 7,238,728 | July 2007 | Seymour | 518/700 |
| 7,302,903 | December 2007 | Behrens | 114/264 |

U.S. Patent Application Publications

| | | |
|---|---|---|
| US 2005/0232833 | October 2005 | Hardy, Coffey |
| US 2006/0211777 | September 2006 | Severinsky |
| US 2007/0142481 | June 2007 | Steynberg et al |
| US 2008/0023338 | January 2008 | Stoots et al |

### SUMMARY OF THE INVENTION

The simplified flow diagram depicted in FIG. 2 will be used here to present an overview and system summary. Preheated water 121 is fed into the alkaline electrolyzer 123 that is powered by renewable electricity 122 to produce high-pressure oxygen and hydrogen. Operating the electrolyzer at very high pressure is the first key requirement for substantial system efficiency gains. The pressurized O₂ and H₂ are then optimally expanded before being used. The source hydrogen, at ˜4 MPa (near term), further heated using waste heat, is then expanded in turbo-generator 125 to ˜1 MPa. The cleaned, source CO₂ is heated and expanded in turbo-generator 126. Both gases are then further heated 127 before being fed into the RWGS reactor 128.

The second key advance is efficient RWGS performance, and two viable approaches (denoted as “multi-stage RWGS” and “recycle RWGS”) are disclosed. As the RWGS products 129 include a lot of water, ultra-high-performance gas-to-gas recuperation is central to either approach. A crucial advance in gas-to-gas recuperation is disclosed in a co-pending patent application. To drive the reaction equilibrium to the right, most of the water must be efficiently condensed out 130 as the reaction progresses. FIGS. 2 and 3 are somewhat more representative of Multi-stage RWGS than of Recycle RWGS, though the latter should ultimately be preferred. In the recycle case, a CuAlCl₄-aromatic complexing method is used to also separate the CO and drive the reaction even farther to the right. If there is excessive CO₂ in the RWGS products, it needs to be recycled 132. The CO and H₂ from the RWGS reactor are then compressed in turbo-compressor 133 to produce the pressurized “new syngas” 134, with typical molar-% compositions as noted in FIG. 2. This is combined 135 with the preheated recycled syngas 147 and fed into the FTS reactor 140. A fixed-bed multi-tubular FTS reactor design is shown to have advantages for high-pressure, variable-rate, low-conversion, high-temperature, exothermic reactions, as needed for high yield of mid-alcohols.—

The third key to success is achieving dramatically improved efficiency in handling low-conversion FTS processes by using high-pressure condensers 141 for the initial separations. Further compression 142 to 8-14 MPa may be needed to achieve adequate gas and product separations in cryogenic condensers 143. To achieve adequate FTS-catalyst lifetime, it is necessary to separate much of the WGS-CO₂ 144 from the FTS products for re-conversion to CO in the RWGS reactor. A novel boost-expand separation process is disclosed that requires nearly an order of magnitude less power consumption than common CO₂ separation methods. This is possible partly because of efficient cryogenic recuperation 147 of the cooling capacity in the recycled syngas after its expansion in turbine 146 back to the pressure needed in the FTS reactor. The separation also benefits from advances disclosed in the co-pending recuperator patent application, and it benefits from higher FTS reactor operating pressure—a counter-intuitive discovery.

The fourth key to RFTS is designing a plant that is inherently compatible with operation over a very wide range of mass flow rates. Variable-angle nozzles, variable-speed motors and generators, and turbine switching assist to this end, along with the use of optimal heat transfer processes. Numerous additional features further improve efficiency, including a refrigeration cycle utilizing the free compressed oxygen, a dual-source organic Rankine cycle heat engine, as disclosed in a co-pending patent application, and an improved CH₄ separation process, as discussed elsewhere.

A fifth key aspect is that local upgrading can be handled more efficiently because of the absence of troublesome impurities in the crude products and because of the availability of abundant hydrogen, oxygen, low-grade waste heat, electrical power, and excess cryocooling capacity. Other beneficial aspects of the separations processes allow simplified recovery of all flash gases and avoid the need for any significant purge stream.

### BRIEF DESCRIPTION OF THE DRAWINGS

FIG. 1 presents an overview of the liquid wind fuels system concepts.

FIG. 2 is a simplified flow diagram of the novel RFTS plant as presented in the Summary.

FIG. 3 is an overview flow diagram of a representative recuperated 240 MW RFTS process.

FIG. 4 is a schematic diagram showing a multi-stage heat-react-condense RWGS process.

FIG. 5 is a schematic diagram showing a recycle RWGS process using CO separation.

FIG. 6 is a flow diagram of a method of using compressed oxygen for cryogenic refrigeration and electrical power generation.

### DETAILED DESCRIPTION OF THE PREFERRED EMBODIMENT

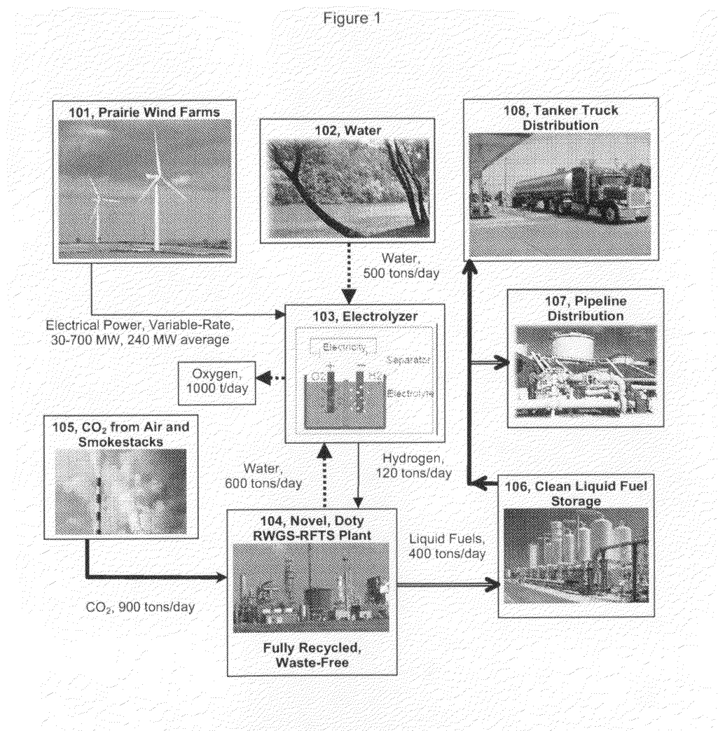

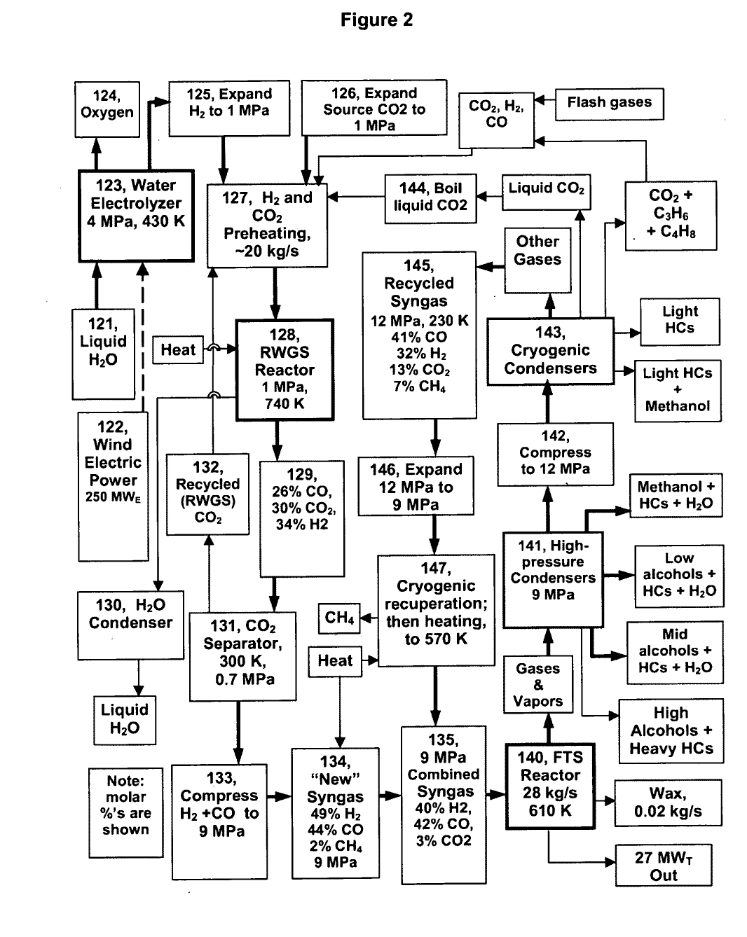

A concept overview was shown in FIG. 1 and described previously in the introduction to the background section. The simplified flow diagram depicted in FIG. 2 and described in the above Summary of the Invention provides a slightly more detailed RFTS plant synopsis.

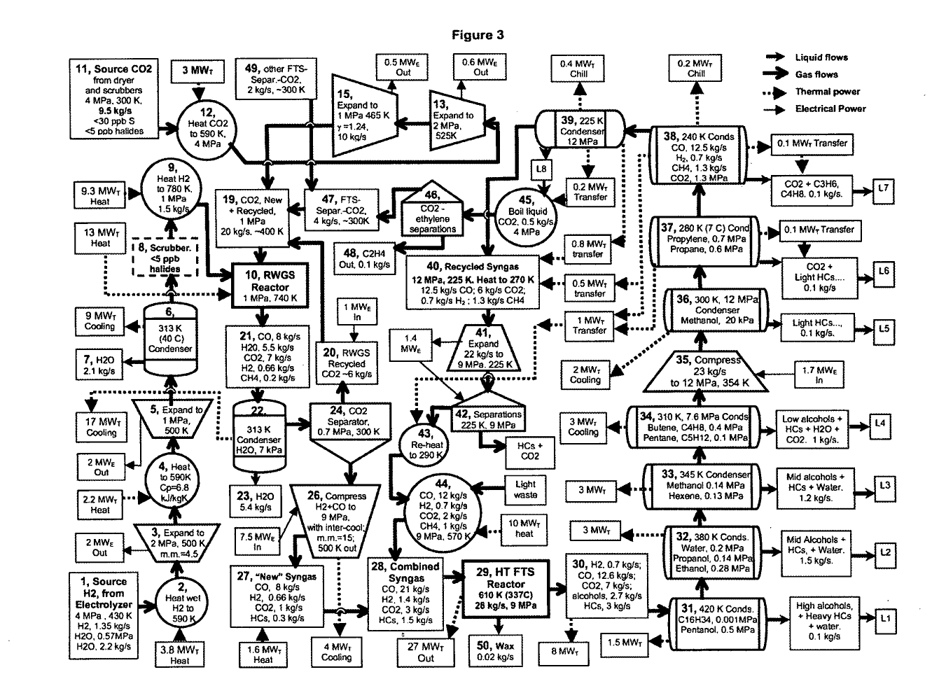

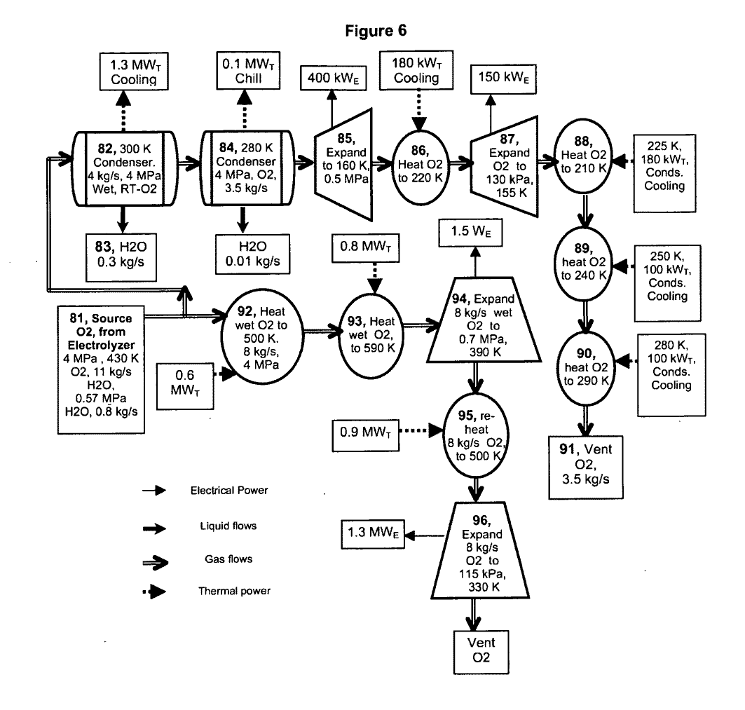

FIG. 3 presents a much more detailed diagram of the RFTS plant showing approximate power and mass flows in the main processes for an example mid-alcohols plant driven by about 240 MW of mean renewable electrical power. (About 5-10 MW more power to the electrolyzer comes from waste heat engines, as will be seen later.) This product mix is chosen here to illustrate that mid-alcohols, which offer significant safety, environmental, and end-use efficiency advantages compared to gasoline, can be produced at higher efficiency than is currently achieved in the best GTL diesel plants. To make it easier to confirm the validity of the analysis and to better address system optimization issues, all of the essential components of the main FTS processes are kept on a single page, and the information-poor symbols historically used in process diagrams have been augmented with more informative text and data. The use of different line styles for heat flows, gas flows, liquid flows, and electrical power flows also helps. Note that thermal-power flows are designated with a subscript T, while electrical power flows are designated with a subscript E. In this specification, the subscripts may be omitted where the context is clear. The handling of the 11 kg/s of pressurized, warm O₂ also generated in the electrolyzer at 4 MPa, 430 K, is not included in FIG. 3. It is discussed later with reference to FIG. 6, where two subsystems associated with the oxygen generated by the electrolyzer are presented in more detail.

The Sources. The RFTS H₂ source 1, 1.35 kg/s, is assumed to be saturated with water, coming from a 430 K, 4 MPa, KOH water electrolyzer. This rate represents 96% of the H₂ from a 250 MW electrolyzer of 80% HHV efficiency—a mid-term objective. (The remaining 4% of the hydrogen is assumed needed for other purposes, and the mechanical energy in the gases is not normally included in the electrolyzer efficiency rating. Also, about 10 MW_{E} of the power to the electrolyzer comes from waste heat engines, as will be seen later.) Of course, older technology electrolyzers, with HHV efficiencies in the range of 66-74%, could also be used, but with higher electrical input requirements. (Steam electrolyzers seem unlikely to compete in practical applications with very-high-pressure liquid-water electrolyzers—probably soon at pressures above 10 MPa, some reasons for which will become clearer later.)

Assuming the electrolyzer pressure is greater than the RWGS operating pressure (as would normally be the case), it's best to first use the wet H₂ in a small heat engine 2, 3, 4, 5 rather than start by drying it, since there's plenty of waste heat at the FTS reactor temperatures. Assuming a source pressure of 4 MPa and an RWGS reactor entry pressure of 1.0 MPa, the H₂ needs to be expanded by a pressure ratio of 4.

Efficiently expanding a very light gas (here the H₂O molar fraction is ˜15% and the mean molecular mass is ˜4.5) by a pressure ratio of just 4 may still require four turbine stages to get 88% expander efficiency (a practical limit at this flow rate and molecular mass). The expander and compressor turbines would probably be optimized for highest efficiency at mean conditions, but they also may need to perform at three times this power during strong winds and at less than one-third mean mass flow rate during calms. If the pressure ratio is adjusted roughly in proportion to the square root of the mass flow rate with optimum speed control, multi-stage expander turbines with fixed nozzles typically see their efficiency drop by about 8% as the mass flow rate deviates from optimum by a factor of three in either direction. Multi-stage compressor turbines with fixed nozzles may see about 15% drop in efficiency for such changes in conditions. However, pressure ratios will not be able to change as much as needed in response to changes in mass flow rates over a wide range in conditions, so it will be necessary to include variable-angle stator nozzles (or parallel turbine switching, as discussed later) to greatly improve efficiency over a broad range of flow rates and pressure ratios. This is not difficult to implement in these turbines and compressors, as they are not required to operate at very high temperatures. Still, it may not be cost effective to aim for 88% hydrogen-expander efficiency initially—perhaps 85% would be more cost effective in a 250 MW plant. (Note that 80% would be more practical in an RFTS plant one-tenth this size. And again, we remind the reader that we consistently refer to average power rather than the less useful peak ratings that the wind and solar industries have normally used.)

An electrical generator efficiency of 94%, a turbine of 85% polytropic expansion efficiency, and one reheat stage 4 (mid-way) are assumed here. The wet H₂ ends up at 500 K, 1.0 MPa before going to the ambient-temperature condenser 6 (313 K may be sufficient, as the hydrogen doesn't need to be very dry). In the process, 4 MW of electrical power is generated at the expense of about 6 MW of waste heat from the FTS reactors into exchangers 2 and 4. The wet H₂ is then cooled and dried in a multi-stage condenser 6 (though shown here only as a single stage, for simplicity) so about 1 MW of mid-grade heat can be recovered before condensing out the water 7 with another 8 MW of cooling (from a cooling tower), which also carries away most salts.

Many fresh-water sources will have NaCl content in the 10-100 ppm range. Even if the electrolyzer source water is of typical single-distilled quality (electrical conductivity of ˜2 μS/cm), its NaCl content may be about 1 ppm. The salts in the source water will concentrate by many orders of magnitude in the electrolyzer and eventually some will be carried through in the gases. The easiest way to help keep the halide impurity in the hydrogen below the 5 ppb level desired for the reactors is to continually bleed concentrated electrolyte from the electrolyzer and steadily add KOH at the required make-up rate. (This will also minimize degradation of the electrolyzer.) If the halide content in the H₂ gas is still above 5 ppb it can be scrubbed 8.

The H₂ then needs to be heated to the RWGS feed temperature, which here is assumed to be about 780 K and for brevity is shown here as a single exchanger 9. In practice, this 9 MW total heating would normally involve 3 to 6 heat exchangers to permit more efficient utilization of first low-grade heat (˜400 K), then low-mid-grade heat (˜500 K), then high-mid-grade heat (˜600 K, FTS reactor heat), and finally high-grade heat (˜800 K or higher, combustion products or electrical heating). A sequence of low-mid-grade and high-mid-grade heat is also appropriate for the initial H₂ exchanger 2, though the re-heat exchanger 4 would utilize mostly high-mid-grade waste heat. The very high thermal conductivity of H₂ helps reduce the cost of these heat exchangers. The hot, dry H₂ then goes to the RWGS reactor 10.

The recovered (source) CO₂ 11 should be delivered at the commercial standard of 99.5% purity. The major impurity is often H₂O, so the CO₂ may need drying first to improve the effectiveness of the subsequent scrubbers. The ratio of source CO₂/H₂ will depend on the product mix. For the mix assumed in this example, 9.5 kg/s CO₂ is about right for ˜1.4 kg/s H₂. To maximize the lifetime of the FTS and RWGS catalysts, sulfur and halides may need to be scrubbed—possibly using molecular sieves. Remaining impurities below the 0.1% level (H₂O, Ar, N₂, CH₄, CO, C₂H₆, He, etc.) don't matter, as shown later. The pressurized CO₂ could be used to provide some of the cooling power needed in the cryogenic separation of the FTS gases, but there are better ways to do that, so instead it may be best to first use it in another heat engine 12, 13, 15 (for the same reasons as for the H₂ heat engine).

The variable-nozzle turbines for a CO₂ heat engine 13, 15 are much less expensive and more efficient (88% should be cost effective) than for the H₂ heat engine 3, 5 though the CO₂ heat exchangers 12 are more costly than the H₂ exchangers 2, 4, 6. The moderate pressure (MP) CO₂ exiting the heat engine 15 will be combined with other recycled CO₂ that will probably be closer in temperature to 310 K. Therefore, re-heat—as shown in the H₂ heat engine 4—is not desired here, since efficiency is improved by minimizing the temperature differences when different streams are combined. The total costs are about the same for each heat engine—probably about $500/kW_{E} for small-scale production quantities (several per year), and about $100/kW_{E} in large-quantity production.

This source CO₂ will be augmented by unreacted CO₂ separated from the RWGS output 20 and by recycled CO₂ 47 separated from the FTS product stream. There may be about 4 kg/s of recycled CO₂ 47 from the FTS products, some of which may be available at 2-4 MPa, which would allow it to be injected into one of the pressurized CO₂ heaters 12, 14 for increased expander output power. However, it is likely that enough ethylene co-production could be achieved in the FTS to warrant separation and sale of this very valuable co-product. If so, it would not be well separated by the primary cryogenic separation process (discussed later) from the pressurized FTS CO₂, so a subsequent CO₂/ethylene separation process would be needed, which would probably result in more of the separated FTS CO₂ being available for RWGS recycling at a pressure too low for expansion through the CO₂ heat engine. The CO₂ heat engine would use only about 1.3 MW of high-mid-grade waste heat (that would not otherwise be needed to heat the CO₂ to the RWGS temperature) to generate about 1.1 MW of electrical power. (The surprisingly high efficiency is because the source gas is supplied pressurized, and the mechanical energy in the source gas has been ignored, a point we further clarify later.) The combined CO₂ streams 19 are then sent to the RWGS reactor 10.

The temperatures and pressures shown for the feed H₂ 9 and feed CO₂ 19 are for example purposes only, especially since the RWGS reactor block 10 represents a choice of complex processes, as discussed in the next section. It is also possible that the RWGS reactor pressure could be as high as the H₂ source pressure, in which case the source H₂ heat engine would be eliminated. Also, some electrolyzers have operated at pressures as low as 0.3 MPa, though their efficiency has been rather low. If the source H₂ pressure is less than the RWGS pressure, a compressor would be required between the source and the RWGS reactor.

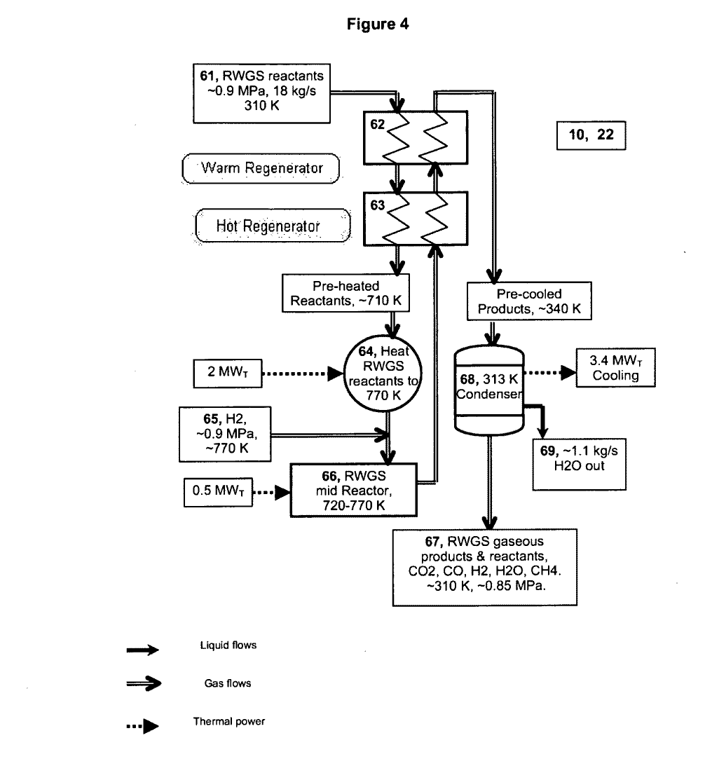

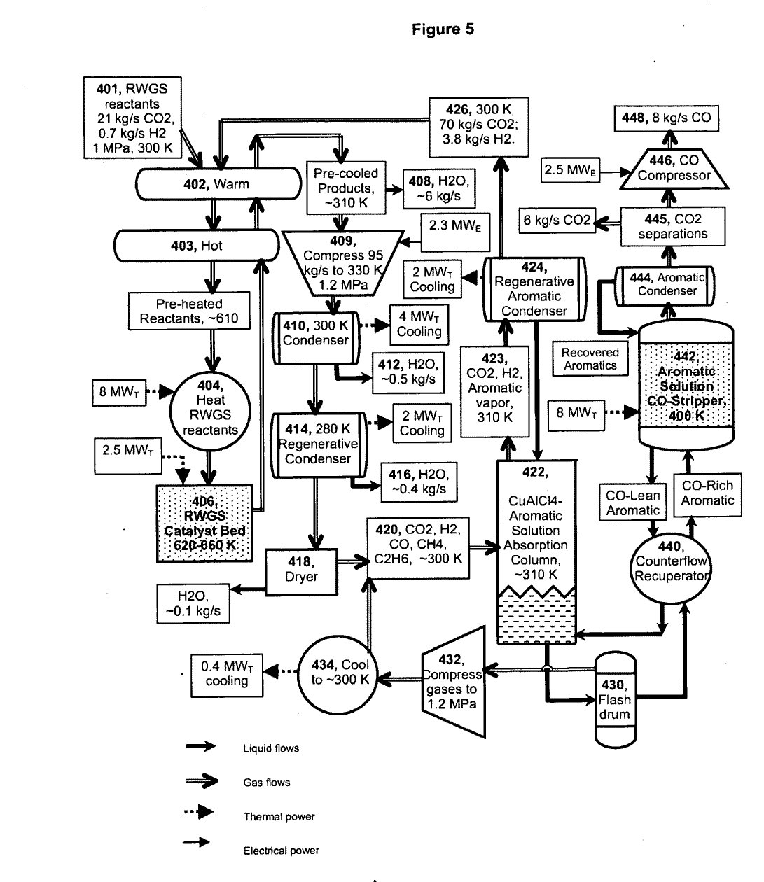

The RWGS Reactor System. The RWGS reactor is shown in FIG. 3 as a single block 10 with a single heat input and a single condenser 22 for simplicity in this system diagram. While a fraction of the heat needed for the RWGS reaction (eq. 8) can be supplied by combustion products directly to the reactors, the requisite low gas velocities in the reactors make it easier to supply most of the needed heat in separate exchangers. Also, it is essential to condense water from the products as the reaction progresses, and it may be desirable to separate the CO. Two different options, first Multi-stage RWGS with just H₂O separation, and then Recycle RWGS with both H₂O and CO separation, will be shown shortly in more detail in FIGS. 4 and 5.

Table 1 presents a summary of data for some relevant conditions. The H₂O partial pressures during the reactions were not reported, but it appears they were very low and the H₂O listed includes that condensed. Other data show Cu/γ-alumina catalysts to be very effective (100% CO selectivity, no methanol or CH₄) for operation above 627 K at 0.1 MPa with H₂/CO₂ ratio of 4, but a higher pressure is required here. Some less successful experiments have had objectives and constraints that have shifted their focus from what is needed for the primary objective here—efficient production of CO from CO₂ with low production of CH₄ and low carbon deposition at moderate temperatures to minimize exchanger, reactor, and catalyst costs. It should be noted that, except in one of the cases listed, there was no attempt to achieve high space velocity (most were around 700 ml/hr/g). Other data suggest the normalized space velocities (gas hourly space velocities, GHSV, cm³/h/g-cat, normalized to STP, 0.1 MPa, 273 K) could be much higher with relatively little adverse affect on the CO₂ to CO conversion.

Keeping the H₂ partial pressure under 0.2 MPa appears to keep the methane low up to at least 720 K. Acceptable performance can be achieved with either Cu/alumina or Cu/silica (and possibly with Cu/SiC) catalysts at over 0.8 MPa total pressure at reasonable space velocity (probably over 5000 ml/hr/g-cat, STP) as long as proper provisions are made. A high H₂/CO₂ feed ratio improves CO₂ conversion, but the ratio must be limited to minimize methane production.

For operation above 700 K, which seems essential for practical CO yield if only H₂O is separated as the reaction progresses, an Fe₃O₄/Cr₂O₃ catalyst may be preferred. It is less active, but it is quite inexpensive, highly resistant to sintering, and has very low methanation activity if properly prepared. It may allow pressures as high as 5 MPa.

TABLE 1
RWGS Experimental Data.

| | | | feed | space velocity, | | | | | | |
|---|---|---|---|---|---|---|---|---|---|---|
| Press | Temp | | ratio | STP | Product Composition (Mole %) | | | | | |
| MPa | K | Catalyst | H2/CO2 | ml/hr/g | H2 | CO2 | CO | CH4 | H2O | CH3OH |
| 6 | 550 | Cu/SiO₂ | 4 | 90,000 | ~50 | ~10 | ~15 | 0.1 | ~20 | ~5 |
| 0.63 | 600 | Cu/γ-Al₂O₃ | 1.88 | low | 32.4 | 3.4 | 31.1 | 0.7 | 32.4 | 0 |
| 0.91 | 608 | Cu/γ-Al₂O₃ | 1.98 | low | 26.1 | 1.9 | 30.4 | 3.7 | 37.9 | 0 |
| 1.08 | 630 | Cu/γ-Al₂O₃ | 1.49 | low | 14.1 | 1.7 | 37.9 | 2.8 | 43.5 | 0 |
| 0.63 | 640 | Cu/γ-Al₂CO₃ | 1.64 | low | 20.2 | 0.6 | 36.9 | 1.8 | 40.5 | 0 |
| 0.91 | 640 | Cu/γ-Al₂O₃ | 1.98 | low | 26.9 | 1.3 | 31.3 | 3.1 | 37.5 | 0 |
| 0.63 | 650 | Cu/γ-Al₂O₃ | 2.3 | low | 35.6 | 0.7 | 28.9 | 2.0 | 32.9 | 0 |
| 0.94 | 650 | Cu/γ-Al₂O₃ | 1.5 | low | 19.5 | 4.3 | 35.4 | 1.8 | 39.0 | 0 |
| 0.46 | 670 | Cu/γ-Al₂O₃ | 1.04 | low | 2.1 | 2.3 | 46.6 | 0.8 | 48.2 | 0 |
| 0.5 | 670 | Cu/γ-Al₂O₃ | 1.44 | low | 7.9 | 1.0 | 39.3 | 4.2 | 47.6 | 0 |
| 0.57 | 670 | Cu/γ-Al₂O₃ | 1.5 | low | 10.8 | 1.1 | 38.4 | 3.8 | 45.9 | 0 |
| 0.7 | 704 | Cu/γ-Al₂O₃ | 2.5 | low | 5.4 | 0.9 | 19.8 | 18 | 55.9 | 0 |
| 0.36 | 718 | Cu/γ-Al₂O₃ | 1.29 | low | ~0 | 0.5 | 42.6 | 4.8 | 52.2 | 0 |

In spite of the fact that the water gas shift reaction has been extensively studied and employed for the past century, there remains considerable discrepancy in published equilibrium constants K_{P} for equation 8 (RWGS reaction) for the temperature range of interest at moderate pressures—i.e., in the 0.3 to 3 MPa range. Pressure has a small effect on eq. 8 (though not negligible, because of the very high polarity of H₂O), but a large effect on eqs. 9 through 13. Recently published values for K_{P} for eq. 8 range from 0.11 to over 0.24 at 700 K. The correct value is probably close to the low end of this range, but a small effective increase is required because of the fugacity coefficients.

Multi-stage RWGS. One stage of one possible RWGS multi-stage reactor design, in which only water is separated as the reaction progresses, is shown in FIG. 4. The RWGS reactants 61 would come from the output of a similar preceding stage at about 310 K, though the temperature entering the first stage may be much higher, as indicated at 19. The reactants are pre-heated against the products to the extent practical using counterflow heat exchange, possibly two rotating honeycomb regenerators in series, 62, 63. While all of the available CO₂ 19 would be injected into the first stage (to drive the RWGS equilibrium to the maximum extent) and be partially converted in each successive stage, only a fraction of the H₂ 9 may be injected 65 into each stage, preferably where shown after final heater 64, to limit CH₄ production. Sending all the source hydrogen into the RWGS reactor, as shown in FIG. 3, gives an initial H₂/CO₂ molar feed ratio near 2 for most fully-recycled FTS processes, and the H₂/CO₂ ratio increases as the RWGS reaction progresses. With current catalysts, this is likely to produce more methane than desired, so staged H₂ injection with some bypass may be better.

The hot reactants and products (CO₂, H₂, CO, H₂O, C₂H₄, C₂H₆, and CH₄) then go to a thin RWGS catalyst bed 67 for partial RWGS reaction. If the RWGS reactor is nearly adiabatic, about 10-20% (depending on the amount of excess H₂, amount of exothermic CH₄ production, etc.) of the CO₂ can be converted to CO for a 50 K drop in the gas temperature—perhaps near the maximum allowable for optimum reactivity control. However, the gas temperature drop can be reduced by providing more of the heating directly into the RWGS reactors 66 (by a network of tubes within the RWGS reactor beds or by external heating of tubes containing the catalyst). This helps to improve temperature uniformity throughout the reactor, especially if a liquid is used for this heat transfer. The reactor products are then cooled against the input stream in one or two regenerators 62, 63 in preparation for H₂O separation in ambient-temperature condenser 68.

The power for final heating 64 and condensing 68 and the flow rates for H₂ injection 65 and H₂O condensation 69 depend on the number of stages used. Without CO separation and with reasonable temperatures, at least four stages are needed to achieve sufficient CO₂ conversion, which is equilibrium limited per stage. More likely, 5 to 10 stages will be desired, and perhaps enough stages could be used to allow sufficiently high CO₂ conversion to eliminate the need for CO₂ separation and recycle after the final stage, though it is assumed needed at 24 in the system diagram. In FIG. 4, it is assumed that the only intra-stage separation is H₂O condensation.

For this example size, the total H₂O yield would be about 5.5 kg/s, so the mean per stage, assuming 5 stages, would be about 1.1 kg/s, which suggests a mean of about 0.12 kg/s H₂ injection per stage. For a practical conversion ratio, the H₂/H₂O molar ratio at the exit of each reactor should be greater than 5, here implying a minimum excess H₂ of about 0.6 kg/s. Of course, RWGS activity is much higher than mean for the first stage, and much lower for the last stage, as the CO steadily increases and excess CO₂ decreases. This can be partially offset by increasing the excess H₂, though CH₄ production will increase from the first stage to the last.

It is useful to look at some typical mid-stage parameters for the 250 MW example assuming five RWGS stages. For example, entry mass composition at 61 might be: 72% CO₂, 21% CO, 5% H₂, 0.5% H₂, 0.5% C₂H₆, and 0.8% CH₄. (The ethane comes from hydrogenated ethylene from the recycled FTS CO₂.) This entry mixture specific heat is about 1.7 kJ/kg-K (at 0.9 MPa, mean regenerator temperature of 480 K), mean molecular mass is about 20, and total flow is about 18 kg/s. Assuming an injection of 0.12 kg/s hot H₂ after final heater 64 and production of 1.1 kg/s H₂O in the RWGS reactor 66, the exit mass composition from the reactor is 57% CO₂, 31% CO, 4.3% H₂, 6.7% H₂O, 0.5% C₂-H₆, and 1% CH₄. (Approximate molar fractions are 0.26, 0.22, 0.43, 0.075, 0.003, and 0.012 respectively.) Compared to the entry composition, the specific heats and mass flow rates are very similar. Hence, the temperature drop in the reactor-output stream through the regenerator is about the same as the rise in the source stream. The mean difference between the two streams depends on the regenerator's effectiveness, which could be about 97%, suggesting a mean temperature difference of about 10 K at the hot end.

The 50 K gas temperature drop in the reactor provides 1.6 MW of the needed 2.4 MW for production of 1.7 kg/s CO. Exothermic methane production generates over 0.3 MW, and viscous effects contribute about 50 kW. Hence, about 0.5 MW of additional high-grade heating (from combustion of methane) must be supplied to the reactor 66. The 60 K of high-grade final heating 64 needed after the regenerators requires about 2 MW from methane combustion.

The additional H₂ injected into each stage could be done at any point, but the location shown seems best, as excessive H₂ partial pressure in final heater 64 could increase methane production there if its surfaces have some catalytic activity. If methane production can be sufficiently limited by optimization of heater surfaces and RWGS catalyst, H₂ injection may be increased to increase equilibrium CO yields. Otherwise, a fraction, possibly about one-fourth, of the source H₂ from 8 would not be used in the RWGS reactor and thus would go directly to the new syngas compressor 26—and bypass the hydrogen heater 9, the RWGS reactors 10, and CO₂ separator 24.

The above mid-stage reactor example was shown operating at ˜740 K to achieve sufficient CO₂ conversion. The earlier stages could operate at lower temperatures and the later stages at higher temperatures. In fact, the CO content entering the first stage is zero, so it could operate at a temperature low enough, possibly as low as 550 K, to be partially driven by heat transferred from an HT-FTS reactor.

It is possible that the economically optimum number of RWGS stages will result in considerably less or more CO₂, perhaps even by a factor of two, remaining in the output from the final RWGS stage than the 7 kg/s suggested in 21 (equal to the sum of that in 20 and 27). Such would have only minor effects on the details of the example analysis presented above for a typical mid-stage, and the effects on the CO and H₂O production shown in 21 are minor, as they are mostly determined by the sources and the recycled FTS-separated-CO₂, shown in 47, assuming most of the CO₂ gets separated in 24 from the other products. The maximum molar fraction of H₂O in the products from the RWGS reactor would preferably be under 0.1.

Recycle RWGS. The combination of CO and H₂O removal from the RWGS products may allow the RWGS reaction to work adequately below the FTS reaction temperature, and that benefit should more than offset the various costs associated with CO separations. Another benefit of CO separation is that it dramatically reduces both CH₄ and C production. The rates for the reverse of eq. [6]—the dominant path for CH₄ production if CO is not very low—and eq. [12] (a significant deactivation mechanism) are both probably second order in CO partial pressure.

Several methods have been demonstrated for the separation of CO from mixtures containing large amounts of H₂ along with various amounts of CO₂, H₂O, CO, CH₄ and inerts. The cryogenic distillation methods of Billy et al in U.S. Pat. No. 6,178,774, work well only for separation of CO when the CO₂ content is very low and there is no desire to reclaim this gas at high pressure. Related restrictions appear to limit the utility of membrane methods and CO adsorption methods based on molecular sieves, though perhaps advances could make these methods competitive.

The most widely used method of CO separation from complex mixtures is the COSORB method of Kinetics Technology International (KTI, originally developed by Tenneco Chemicals). This process uses a solution of CuCl and AlCl₃ in equal molar amounts in toluene (n.b.p.=384 K) for the selective absorption of CO from mixtures containing CO₂, H₂, CH₄, and inerts. The Cu(I)—CO complex is formed at about 290-320 K and moderate total pressures (0.2-3 MPa), and the CO is released at about 370-420 K and low pressures (0.1 to 0.5 MPa). Many other related CO-absorption solutions, generally with two benzene rings, such as 1,2-diphenylethane (bibenzyl, n.b.p.=546 K), 1,3-diphenylpropane (n.b.p.=572 K), and diphenylmethane (n.b.p.=537 K), are disclosed by Hirai et al in U.S. Pat. No. 4,460,384. Although CO solubility is lower, they offer two or three advantages: much lower vapor pressure, much better complex stability against moisture in the gas, and probably less sensitivity to deviations in the molar ratio of AlCl₃ to CuCl. Among the diphenyl solvents, only diphenylmethane is currently available at a price that could be considered for a separation process, though the others would also likely be produced at a practical price with sufficient market demand. Other salts have also been used successfully, including CuMgCl₃.

FIG. 5 illustrates an implementation of a CO-separation process with the RWGS process. This essentially performs the function of the blocks labeled 9, 10, 20, 21, 22, 23, 24 in FIG. 3. Ideally, the feed in 401 would be an approximately stoichiometric mixture of CO₂ (from source 15 and FTS-separated 47) and the H₂ needed for the RWGS reaction, minor CH₄ production, and hydrogenation of C₂H₄ (from the FTS-CO₂). With full H₂ and CO₂ recycle, the following may be a typical net RWGS reaction (assuming limited C₂H₄ and CH₄ separation from the FTS-CO₂):

39CO₂+43H₂+H₂O+CH₄+C₂H₄ [symbol] 38CO+41H₂O+2CH₄+C₂H₆ [14]

In practice, a significant amount of CO₂ and a little H₂ leaves the loop with the CO and HCs, so the H₂ needed and CO produced are both somewhat less than the above suggests and the input CO₂ is greater. Production of ˜8.5 kg/s CO produces ˜6 kg/s H₂O and ˜0.15 CH₄. A preliminary COSORB simulation indicates that ˜7 kg/s CO₂ would leave the loop with the CO, so about 21 kg/s CO₂ and 0.7 kg/s H₂ is needed at 401. The balance of the source H₂ from 8 would go directly to the syngas compressor 26.

The source reactants 401 are mixed with the recycled reactants 426 and warmed against the RWGS products in regenerators 402 and 403, similarly to that seen earlier in FIG. 4. However, the flow rates now are much higher. Assuming a final catalyst temperature of 620 K in the RWGS reactor bed 406 and negligible CO, CH₄, and H₂O in the recycled reactants, the molar fractions of CH₄, CO, and H₂O leaving the RWGS reactor may be about 0.01, 0.08, and 0.09 respectively. Since less than one-sixth of the reactants can be converted per pass at this temperature, the recycled reactants 426 would be at least five times that of the source reactants. Hence, the total mass flow rate through the reactor 406 and each side of regenerators 402 and 403 would be at least 90 kg/s.

In the process shown in FIG. 5, the only method for removing the CH₄ from the recycle loop is the stripper 442, which is not very effective at this task. Thus, the equilibrium molar fraction will build to a much higher level than assumed above—to the point that the CH₄ flow rate leaving in the stripper overhead balances the sum of that in the source stream 401 and that produced in the reactor loop. Hence, barring a more effective method of CH₄ separation, it is important to keep its production rate in the RWGS reactor and regenerators very low. With the COSORB process, it appears that the equilibrium CH₄ molar fraction in the primary RWGS loop would be in the 8-20% range, which is certainly quite acceptable, though not insignificant. Of course, radically different ratios of H₂/CO₂ in the recycled reactants would also work, and lower ratios should keep CH₄ production lower. With no significant removal of H₂ from the recycle loop other than the RWGS reaction itself and no substantial storage, the loop ratio can quickly change in response to minor changes in the feed ratio, so careful control of the feed ratio is necessary.

As the RWGS heat of reaction is about 1.4 MJ/kg of CO, an 8.5 kg/s CO production rate requires about 12 MW_{T}. The assumed 0.13 kg/s CH₄ production (257 kJ/mol for the dominant reaction) and the ethylene hydrogenation together provide about 2 MW_{T} of heating. If 4 MW_{T} is transferred directly into the reactor, the remaining 6 MW would need to be provided from the temperature drop in the reactants and products. For the typical mixture here, C_{P}=1.8 kJ/kg-K at 640 K; so, with minor heat losses, a gas temperature drop in the reactor of 40 K is sufficient, though more is needed if less heat is transferred directly into the reactor. Assuming about 97% effectiveness in the regenerators 402 and 403, the pre-heated reactants leave the hot regenerator 403 at 10 K below the temperature of the products leaving the RWGS reactor; hence, ˜50 K of heating, or ˜8 MW_{T}, is required in exchanger 404.

Note that we have assumed the reactants 401 are supplied at 300 K. Hence, the 9 MW of hydrogen preheating 9 shown in FIG. 3, as needed for the RWGS method of FIG. 4, is no longer needed. Rather, the H₂ and CO₂ preheating are essentially all provided by the massive regenerators 402, 403, which transfer ˜40 MW of heat from the products to the reactants at a temperature difference of ˜10 K. As the vapor pressure of water at the product exit temperature of 310 K is only 6 kPa, most of the water condenses in the “warm” regenerator 402 before it gets to the “condenser” 410, and this must be taken into account in the warm-regenerator design (as indicated at 408).

The best place for compressor 409 (required to make up the pressure drops in the various components in the recycle loop of FIG. 5) is prior to the 300-K water condenser 410. For a total pressure drop of 0.2 MPa, the electrical power required here would be about 2.5 MW_{E}. A 300-K condenser 410 removes additional water, and a 280-K regenerative condenser 414 may be needed to remove more residual water. Even without condensation, cooling 90 kg/s of this rather dry gas by 25 K requires over 3 MW_{T}, though recuperation substantially reduces the required cooling power. Further drying 418, perhaps using desiccants such as activated alumina or silica gel, is needed with the COSORB process, but probably not with some other aromatic solutions. The dry H₂—CO₂—CO—CH₄ mixture 420 (molar fractions about 0.37, 0.4, 0.09, 0.1), along with some other inert gases, then goes into the selective absorption column 422, for slightly exothermic CO-complexing/absorption.

The CO-rich aromatic solution is drawn off the bottom of the absorption column to a flash drum 430. The CO-lean overhead 423 from the absorption column 422 contains solvent vapor, which must be condensed out. As previously noted, a higher boiling aromatic than toluene would simplify the subsequent aromatic reclamation processes, though CO capacity would be less. Under optimum conditions, the CO molar fraction in 423 may be below 0.01, but CO molar fractions of 0.05 are acceptable. Using a recuperator or regenerator 424 here dramatically reduces the cooling power required. Several methods of efficiently obtaining the substantial cryogenic cooling needed in the main FTS separations loop are disclosed later. Excess cooling may be generated by those methods to provide the cooling needed here, or other methods may be utilized. With a high-boiling aromatic, the electrical power requirements for the cooling can be below 2 MW_{E}, but the details of the reclamations will depend heavily on the choice of solvent and pressures.

Much of the CO₂, H₂, and inerts physically absorbed (dissolved) in the aromatic solution will come out in the flash drum 430 as the pressure is reduced from about 1 MPa to perhaps 0.5 MPa. The solubility of the HCs above C1 in aromatic solvents is high and the fraction of these HCs in the RWGS products is low, so most will go into the aromatic solution along with the CO. Some HCs will come out of solution in the flash drum, and some will continue to the stripper 442. The amount of gas flashed from the solution will depend heavily on the pressures, temperatures, and complex concentrations chosen. Assuming a total flash gas (mostly CO₂) flow rate of 4 kg/s, the amount of compressor power required in compressor 432 to compress it back to 1.2 MPa for recycling would be under 0.4 MW_{E}, and a similar amount of cooling is then needed in exchanger 434. The compressor power and the low-grade heating required for the flash drum are not shown in FIG. 5. The pump for the absorber solution is also not shown, and it may require ˜0.7 MW_{E}.

The CO-rich aromatic solution from the flash drum is heated against the CO-lean aromatic solution returning from the stripper 442 in a counter-flow recuperator 440. With the COSORB (toluene) process, the solution mass flow rate can readily be less than 70 times the CO flow rate, and possibly much less according to some reports. However, the COSORB process requires thorough drying 418 and substantial cooling for adequate reclamation of the aromatic vapor, which needs to be kept to low levels in the RWGS reactor.

It is important to appreciate the significance of the toluene reclamations if the COSORB process is used, as high gas-flow rates are present in condenser 424 and rather large temperature change is required in condenser 444. Extreme drying as well as costly solvent reclamation can be avoided by using a better choice for the solvent. The absorber-solution mass flow rate using 1,3-diphenylmethane as the solvent (which solves the water and solvent vapor problems, as disclosed in U.S. Pat. No. 4,460,384) may be 150 times the CO mass flow rate; and the viscosity of the solution is much higher. Quite likely, more optimum solutions, such as mixtures of 1,2-diphenylmethane, 1,3-diphenylpropane, and polystyrene, as suggested by some of the experiments in U.S. Pat. No. 4,460,384, will be developed that better fit the circumstances here—where a moderately low water-vapor partial pressure is required anyway for other reasons. Still, it is likely that the amount of heat transfer required in recuperator 440 will be over 100 MW. Liquid-liquid heat exchange is generally much less expensive than gas-gas exchange for a given effectiveness, though here the thermal conductivities of the solutions are rather low and viscosity may be high. However, there is an abundance of low-grade heat available from the electrolyzer, so recuperation effectiveness is not too critical. The exchanger design is somewhat complicated by the fact that there will be quite a bit of gas evolution from the CO-rich solution as it is heated. A novel recuperator design, as disclosed in a co-pending application may be preferred here. With highly effective recuperation, the amount of low-grade heat needed in the stripper should be well under 10 MW_{T}.

The CO leaves the absorber solution in the stripper overhead along with some aromatic vapor, CO₂, HCs, very minor amounts of H₂, and trace amounts of N₂ and other inert gases. The aromatic vapor needs to be condensed out, and again a regenerative condenser 444 can be used to minimize the cooling power required. After aromatic reclamation, some of the CO₂ may need to be separated 445 from the CO (perhaps using amine absorption). The product, mostly CO (and at about 300 K), is then compressed in a multi-stage compressor 446 to the pressure needed for the syngas. (The separated CO₂ is recycled, as shown earlier at 20 in FIG. 3.) Compressing ˜10 kg/s of mostly CO from 0.5 MPa, 310 K, to 9 MPa with a single mid-way intercool (at about 2 MPa) requires ˜2.5 MW_{E} and delivers the product 448 at ˜420 K. This is a rather substantial amount of electrical power. However, an alternative CO separation process (such as membranes or molecular sieves) that requires major expansion and re-compression of most of the CO₂ or H₂ in the RWGS recycle loop could require an order of magnitude more compressor power. This illustrates the importance of avoiding substantial expansion and re-compression within the main RWGS recycle loop.

For efficient RWGS system performance, it is essential that the maximum sum of the partial pressures of H₂ and CO₂ within the primary recycle loop be less than twice the minimum sum of the partial pressures of H₂ and CO₂ within the primary recycle loop—that driven by recycle loop compressor 409. With optimum design of regenerators 402, 403, reactor 406, and CO separator system 422, 424, this key pressure ratio can be below 1.3.

Higher RWGS reactor temperatures would allow lower recycle ratio, smaller regenerators, and higher CO molar fraction in the reactor products. However, achieving CO and H₂O molar fractions each above 0.12 in the reactor products (corresponding to RWGS equilibrium at about 700 K) is unlikely with a copper catalyst. Using an Fe₃O₄/Cr₂O₃ catalyst may still not permit operation above 800 K, as the metallic regenerator or recuperator (discussed shortly) is likely to have some methanation activity. Of course, the CO separation process may leave a significant fraction of the CO in the recycle stream 426. This may lead to reactor product molar fractions up to 0.15 and 0.1 for CO and H₂O respectively at 700 K, or up to 0.2 and 0.15 for CO and H₂O respectively at 820 K reactor exit temperature. Lower RWGS reactor temperatures require larger recycle ratio and larger regenerators. However, less temperature rise in exchanger 404 would be needed, and the heat may then be able to be supplied by the FTS reactor.

As noted earlier, the first stage of the multi-stage RWGS process could operate at a temperature low enough to be driven by an HT-FTS reactor. Hence, one option is to begin the RWGS process with one stage similar to that of FIG. 4 (but at a lower reactor temperature) and follow it with the recycle process of FIG. 5. However, the output from the condenser of even the first stage may contain more CO than should be fed into the reactor 406 of the recycle process. If so, the feed could be injected into the recycle-RWGS loop at 409, for example, rather than at 401.

Miscellaneous RWGS Comments. Returning again to FIG. 3, recall that 10, 21, and 22 summarize typical results of multi-stage RWGS reactors, one mid-stage of which was described in some detail with reference to FIG. 4. With that process, there likely would be enough unconverted CO₂ from the final RWGS stage to require being separated 24 from the final RWGS products, probably by pressure swing absorption (discussed briefly later), and recycled 20 back through the RWGS reactor. The CO and un-reacted H₂ may need to be dried again after a CO₂ separation process.—

It could be more cost effective to operate at higher temperatures and pressures than suggested in FIG. 4 and accept the extra CH₄ rather than strive to achieve very low CH₄ production from the RWGS reactor. Sending the CH₄ through the FTS reactor won't hurt its performance significantly. Removing the CH₄ after the FTS reactor is preferred so only one CH₄ separation system is needed, as the HT-FTS reactor will generate at least another 5%. However, efficient separation of CH₄ from syngas is not easy, so it is important to keep its total production as low as practical.

The water 23 condensed from the RWGS reactors will be very clean, containing only trace amounts of impurities from the recycled FTS-CO₂. Hence, its purification process is extremely simple. This is also true of the water from the H₂ drying 7 and that condensed from the oxygen stream. These three very clean water sources can easily be recycled and would make up about half of the water needed to supply the electrolyzer.

As the vapor pressures and boiling points of ethylene and ethane are close to those of CO₂, they are not well separated from recycled FTS-CO₂ 47 by the simple fractional condensation and flashing processes that are used initially. However, as noted elsewhere, they would normally be mostly separated 46 from the recovered CO₂ by other means (oil absorption, selective adsorbents, membranes, or cryogenic distillation) for sale or reformation. Keeping the level of HCs going into the RWGS reactor low is also beneficial for improving the lifetime of the RWGS catalyst.

The conditions and flow rates indicated for the RWGS reactors in the Figures seem near optimum with current technology. Quite likely, there will be further improvements in the catalysts. It appears unlikely that the desired RWGS reactant temperature would be above 900 K, as it is also desirable to operate at reasonably high pressures and the combination of high pressure and high temperature rapidly increases unwanted CH₄ production and decreases catalyst lifetime.—

High-performance, Cost-effective Heat Transfer. The heat recuperation 62, 63 in the example RWGS single-stage shown in FIG. 4 is over 10 MW. The total recuperation in 402, 403 in the RWGS method shown in FIG. 5 is over 40 MW. Clearly, greater than 80% effectiveness, and preferably more than 95% effectiveness, is critical for competitive operation of the RFTS plant. Doty, in U.S. Pat. No. 4,676,305, discloses a compact method of achieving highly effective recuperation with low pressure drop for moderate-pressure gases. However, this microtube recuperator has not yet been shown to be commercially competitive with the brazed plate-fin type, in wide usage in recuperated open Brayton cycles in the 30-250 kW range. See, for example, the microturbines available from Capstone Turbines Corporation, of Chatsworth, Calif.

Misconceptions persist in some circles that high-effectiveness gas-to-gas exchangers can utilize tubing diameters of 1-5 cm and lengths of 4 to 20 m for one of the gases, as in classic shell-and-tube exchangers, without incurring huge mass and cost penalties. However, optimized compact exchangers require relatively low flow velocities (several percent of the sonic velocity), exchange-flow-path lengths in the range of 0.1 to 2 m, and passage hydraulic diameters (as usually defined) of 0.5 to 8 mm, with the larger sizes corresponding to pressures near 0.1 MPa and the smaller sizes corresponding to pressures above 0.5 MPa. They have also required the use of construction materials which have fairly low thermal conductivity.

An alternative to paralleling millions of microtubes that has seen rather little usage but may be the most competitive for RWGS recuperation is the rotating honeycomb regenerator, as used in some turbine engines. Oda et al in U.S. Pat. No. 4,304,585 disclose an early ceramic design. Regenerators have seen very little usage in recuperated microturbines largely because of the difficulties in obtaining adequate isolation between the high-pressure and low-pressure streams and the shedding of ceramic particles, leading to turbine abrasion. The dynamic sealing problem has been somewhat addressed by a previous collaborator, D G Wilson, in U.S. Pat. No. 5,259,444 for some applications. However, the sealing problems are essentially non-existent in RWGS recuperation, as the pressure difference between the two streams is quite small and minor mixing of the streams is of little consequence.

Ceramic is usually selected for honeycomb regenerators in recuperated aero-turbine applications because of the need for oxidation resistance at high temperatures and the advantage of low thermal conductivity in the flow direction. Rotating ceramic honeycomb regenerators have demonstrated effectiveness above 98%, while the brazed plate-fin recuperators seldom achieve more than 87% effectiveness, primarily because of cost and mass optimization reasons. The honeycomb regenerators can be an order of magnitude more compact and an order of magnitude less costly for a given exchange power and effectiveness than plate-fin microturbine recuperators—which can be an order of magnitude more compact than the gas-to-gas recuperators normally seen in chemical engineering applications.

As oxidation resistance is irrelevant in the RWGS regenerator and temperatures are lower than in turbine exhausts, the RWGS regenerator could probably be made at lower cost and with much higher reliability from a low-conductivity alloy honeycomb, such as silicon bronze, stainless steel, or some magnesium or aluminum alloys, none of which are likely to have high methane or CO selectivity. It is important to appreciate that high CO selectivity here would be detrimental, as the products leave the recuperator at low temperature, where the equilibrium constant for CO production is very low. Methane selectivity is likewise detrimental, and this may establish the upper temperature limit for operation with the Fe₃O₄/Cr₂O₃ RWGS catalyst unless the recuperator surfaces can be adequately deactivated. The thermal conductivity of silicon-nickel-bronze can be below 40 W/m-K, and 120 W/m-K is usually sufficiently low. For example, a magnesium alloy with thermal conductivity about 90 W/m-K has been used experimentally in a helicopter turboshaft engine. Titanium alloys may be better, and it appears that their relative cost will decrease over the next decade. The much higher thermal stress tolerance of metals compared to ceramics is extremely beneficial with respect to durability, as thermal stress is a primary factor limiting regenerator design and contributing to shedding of particles from ceramic regenerators.

The regenerator cost is typically near optimum when pore diameters are about 0.7 mm for mobile gas-gas exchange applications. This small size could lead to excessive back pressure because of surface tension if condensation occurs within the regenerator. Hence, a more preferable arrangement for RWGS recuperation, where the product stream may be saturated with H₂O above 360 K, may be to use two regenerators in series. The one at the hot end could use pore diameters under 1 mm and handle perhaps 80% of the exchange power (i.e., the temperature at the junctions between the two may be about 380 K). The one at the cooler end could have larger pores to avoid plugging from condensation. The relevant design theory, well understood for more than two decades, has recently been reviewed and updated by DG Wilson in “Design and Performance of a High-Temperature Regenerator Having Very High Effectiveness, Low Leakage and Negligible Seal Wear”, paper GT 2006-90096, Turbo-Expo 2006. Pore diameters as large as 8 mm may still be superior with respect to cost and effectiveness to that often seen in chemical engineering applications using conventional shell-and-tube exchangers, which in turn could be more effective than the phase-change approach advocated by Severinsky, as it does not easily permit minimization of irreversibilities (loss in available work, or energy) from large temperature differentials.

There are other places in the RFTS plant where the use of rotating honeycomb regenerators may be beneficial (i.e., where efficient heat exchange is needed between clean gases of little pressure difference and of similar thermal powers), as will be seen. However, a seldom-mentioned limitation of regenerators arises in high-pressure applications—carryover. This may limit the utility of the regenerator in many of the applications in the RFTS plant. A highly advantageous recuperator design for most gas-to-gas and some liquid-to-liquid applications is the subject of a co-pending patent application.

As previously noted, it may be desirable to drive the RWGS reaction by heat transfer from the FTS reactor. Such would require minimal temperature drops, as known catalysts allow for very little temperature difference between the reactions. Water is difficult to use for the exchange medium above its critical point (647 K, 22 MPa). A heat transfer fluid with normal boiling point above 450 K, such as a molten metal alloy, high-boiling organic, or salt may be best for the exchange medium between the reactors, if such exchange is utilized.

It is essential to utilize the higher-grade waste heat as effectively as possible. The source-gas heat engines disclosed earlier permit very efficient utilization of a fraction of this available heat, and some may be used to drive the RWGS reaction. A DORC, described in a separate application, permits efficient utilization of the balance of the waste FTS heat.

“New” Syngas Compression. The mixture of separated CO and H₂ (along with minor amounts of CO₂, CH₄, H₂O, C₂H₆, CH₃OH, etc.) from the CO₂ separator 24 (or the RWGS 10 if the CO₂ separator is not needed) is then compressed using a multi-stage compressor 26, perhaps with inter-cool, to form the “new syngas” 27. This may be heated with waste heat before being mixed 28 with recycled syngas 44 and sent into an FTS reactor 29. The “new” syngas 27 is not all new, as perhaps a third of the CO₂ from which it is made is recycled FTS-CO₂ (47), which explains the fact that the carbon in the “new” syngas stream is greater than the carbon in the source CO₂ stream.

Compressing the “new” syngas mixture from the assumed 0.7 MPa 24 (final RWGS dryer outlet) to the 9 MPa assumed needed in the FTS mid-alcohols reactor is the single most electrical-power-intensive process in the RFTS plant (other than the electrolyzer). The mean molecular weight here is about 15, which makes 88% polytropic compressor efficiency practical, even with variable nozzles. Therefore, by starting from a low temperature (˜310 K) and using intercool midway (at 3 MPa), this compression can be done for about 7 MW_{E} with available turbine technology at a reasonable cost. With no intercool, the required electrical power for this example would be at least 50% higher (primarily because the output ends up at a much higher temperature than desired going into the FTS reactor), but more than one intercool is not justified for a total compression ratio under 20. Partial intercool may be preferred so that the compressed new syngas ends up at the temperature desired for the FTS reactor without further heating.

The Fischer-Tropsch Synthesis Reactors. There are several FTS reactor possibilities suitable for use with the renewable syngas discussed above. Many state-of-the-art FTS reactors are well described in the recent book edited by AP Steynberg and ME Dry, Studies in Surface Science and Catalysis 152, Fischer-Tropsch Technology, Elsevier, 2004. Steynberg et al also skillfully disclose an improvement on a two-stage FTS reactor arrangement in US 2007/0142481, wherein the syngas first partially reacts in a 3-phase LT-FTS reactor and its tail gas (some products and un-reacted syngas) then go to a 2-phase HT-FTS reactor for further reaction. This approach appears optimum for their previously desired balance of mostly lubricants, high-quality waxes, linear alkyl-benzenes (LABs, for soaps), gasoline, diesel, light olefins, and some oxygenates, including mid-alcohols.

Sasol's FTS has already saturated the heavy n-paraffin and LAB markets. Recent and projected market trends suggest more profitable product balances would maximize either mid alcohols or light olefins—at least after satisfying the lesser demand for lubricant base stocks, cyclohexane, and some other petrochemicals. Plant designs to maximize light olefins, based on the Sasol 2-phase HT-FTS reactor, have been described in the above referenced book by Steynberg and Dry. Plant designs to maximize efficient production of mid-alcohols from ultra-clean syngas in high-temperature reactors have not yet been well described. Hence, an approach to such is presented here.

There are five distinguishing features of all of the more successful prior attempts at FTS of mid-alcohols:(1) H₂/CO ratio below 1.4, possibly as low as 0.7; (2) high CO partial pressure, in the range of 2.5 to 10 MPa throughout the reactor; (3) properly promoted catalyst for improved mid-alcohols selectivity at the expense of reactivity; (4) low CO conversion, possibly below 30%; and (5) moderately high reaction temperatures—about 530-630 K, depending on the catalyst and pressure. The conditions and results of the example that follows are best estimates based on published data, though further catalyst developments are likely.

The HT-FTS reactor 29 here is assumed to be operating at 9 MPa, 610 K (337° C.) at mean design conditions. The temperature chosen here is near the mean of that used in the recent highly promising results on K₂CO₃-promoted β-Mo₂C catalyst and that which has been shown to give best selectivity toward mid-alcohols with K/C/Co-promoted MoS₂ catalysts. (While sulfided catalysts would not be used in an RFTS plant, as they exhibit poor long-term stability when sulfur is not present in the syngas, some of their selectivity trends are similar to those of non-sulfided alcohols catalysts.) The pressure chosen is a little above that used in a preliminary K₂CO₃-β-Mo₂C study, as the temperature here is higher. The pressure, temperature, and H₂/CO ratio are also close to those preferred for high selectivity of mid-alcohols from Cu/ZrO₂ catalysts.

The assumed catalytic selectivities on the basis of C-atom-% in this example are similar to those demonstrated recently with a K₂CO₃-promoted β-Mo₂C catalyst: 24% ethanol, 17% methanol, 8.5% propanols, 6% C₅-C₇ olefins, 6% propylene, 6% methane, 4% C₈-C₁₂ olefins, 4% butenes, 3.5% C₁₃-C₁₉ olefins, 3% butanols, 2% ethylene, 2% C₂₀₊, 2% acetone, 1.5% C₄-C₇ alkanes, and lesser amounts of others. Assumed total CO conversion is 28%, plus over 10% WGS. Unreacted reagents are assumed fully recycled, as shown in FIG. 3.

As discussed in more detail in a later section on variable power, temperatures and pressures would be higher than mean conditions during gales, when flow rates increase. During light winds, temperatures and pressures would be significantly lower. At the higher temperatures, the pressure must also be increased, though it is essential that the pressure stay much lower than the vapor pressure of water at the FTS reactor temperature (as explained by Zhang et al in U.S. Pat. No. 7,001,927) and lower than the vapor pressure of the highest boiling product desired in large fraction in the FTS vapor product stream.

The FTS reactor is fed from two streams, assumed to be at 9 MPa and 575 K in this example—the “new” syngas 27 and the recycled syngas 44. Their compositions are different, and the composition of the new syngas may be adjusted as needed (by changing the RWGS CO₂/H₂ feed ratio) to achieve and maintain an optimum H₂/CO ratio in the FTS reactors. Both syngas streams preferably should be pre-heated close to the FTS temperature to minimize thermal gradients in the FTS reactors, but a recycled syngas temperature as low as 420 K prior to mixing may be acceptable in some reactor designs. The recycled syngas heating would preferably utilize a sequence of low-grade, mid-grade, and finally high-mid-grade waste heat.

The composite vapor/gas HT FTS product goes to fractional condensation, and the heavy products 50 go to hydrocracking. The various product flow rates can vary greatly, depending on catalysts and conditions, but the numbers shown in the figures are useful for illustration purposes and for representative efficiency and power calculations.

After the catalysts, the biggest differences between HT reactors optimized for gasoline and those optimized for mid-alcohols are the H₂/CO ratio, the pressure, and the conversion per pass. Better selectivity for alkanes and alkenes is obtained with the H₂/CO ratio fairly close to the stoichiometric ratio—a little less than 2. Better selectivity for alcohols is generally achieved with this ratio close to 1. CO conversion per pass is much lower for mid-alcohols. This means there will be much more CO re-circulating in the mid-alcohols plant than in a gasoline or diesel plant. Another difference is that the mid-alcohols process will usually do better with more CO₂ in the feed stream (this helps suppress WGS activity), and a little of this CO₂ may be converted to products. Still, there would be more WGS activity than in LT gasoline or diesel plants, which means there will be less water in these HT-FTS products and recycling of the H₂ is absolutely essential.

Conventional wisdom has been that lower temperatures and pressures allow higher plant efficiency, so less work has been done over the past 15 years on HT FTS catalysts. However, high pressures actually allow for greatly improved efficiency in CO₂ separations, and higher temperatures permit higher efficiency in reactor waste heat utilization. High temperatures also are essential for high yields of most of the products expected to be the most profitable for the next 15 years—ethylene, propylene, mid-alcohols, gasoline, and butylene.

Most prior demonstrations of mid-alcohols FTS have utilized small, fixed-bed, multi-tubular reactors with the catalyst inside the tubes and the coolant outside. Moderate-diameter tubes (typically 30 to 60 mm, depending on catalyst type) have generally been used. The syngas inlet is at the top, and the heavy products trickle down along with the flowing gaseous reactants and products. Historically, fixed-bed, multi-tubular reactors have generally not been the most cost-effective approach in prior large scale FTS. However, the Sasol 2-phase HT fluidized bed does not appear suitable for mid-alcohols, as it requires that the pressure be low enough (1.5 to 3 MPa) to prevent wetting of the particles. Alcohols are not highly selected except at higher pressures.

It is possible that the preferred large-scale reactor type could be a 3-phase slurry. However, the liquid phase is continuously being hydrocracked in HT slurry reactors, so wax or other suitable liquids must be continuously added. Because some of the unfavorable HT slurry reactor experiences have been at lower pressures than needed to limit the loss of the lighter components of the liquid phase, high-pressure slurries may be suitable at the lower end of the HT range of interest. Some high-stability organics, including tetrahydroquinoline, tetrahydronapthalene, and decahydroquinoline, may be useful as a majority of the liquid phase in a slurry reactor at temperatures up to 650 K, but their suitability remains unproven. It should also be noted that there are considerable cost and technical complexities associated with the cyclones in 2-phase reactors or the particulate filters in 3-phase reactors, and these are avoided in fixed-bed multi-tubular reactors.

The fixed-bed, multi-tubular reactor seems likely to be preferred for mid-alcohols and most other RFTS. There are several strong arguments for the fixed-bed reactor: (1) it is compatible with operation over a very wide range of pressures, flow rates, and temperatures with no fluidization challenges; (2) there is no difficulty with keeping the catalyst from being entrained in the exit flow (which would be disastrous for the turbines needed in the cryogenic separation process); and (3) its engineering design is quite scaleable and predictable. The preferred approach would be to have a larger number of smaller FTS reactors in parallel at the RFTS plant than normally seen in conventional GTL plants, as there is no economic advantage with fixed-bed reactors in going beyond a size that is easily transported by truck.

The fixed-bed reactors have exhibited much lower activity than the slurry and fluidized reactors partly because they have not utilized small tubes. Smaller tubes increase the cost of the tube sheets and tube welding—especially in high-pressure reactors if the coolant and syngas pressures are very different. However, the cost with smaller tubes—about 10 to 30 mm outer diameter—need not increase as much as has been thought, for 3 primary reasons: (1) the catalyst activity can be greatly increased with optimized catalyst supports at higher reactor pressures, (2) advances in robotic welding, and (3) the use of a high-boiling liquid coolant allows the pressure difference between the coolant and the syngas to be kept small over all operating conditions.

Using a larger number of smaller, parallel reactors makes it much easier to deal with variable power, and catalyst maintenance is also simplified. Un-needed reactors can be shut down during light winds for on-line catalyst rejuvenation with no adverse affect on plant operations.

Catalyst lifetime in HT reactors is not as significant an issue as once thought. Coking, which has widely been known to increase as the H₂/CO ratio is reduced, has recently become better understood. At high temperatures, carbon deposition onto the surface occurs predominately through pyrolytic and dehydrogenation reactions. The selectivity toward dehydrogenation occurs when hydrocarbons are adsorbed strongly to the surface, and progress has been made toward reducing this. One route has been the use of a hexa-alumina lattice, which minimizes the formation of large ensembles of active sites that are responsible for strongly adsorbing hydrocarbons. More importantly, carbon deposition is often approximately proportional to ρ_{CO}/ρ_{H2}², (the partial pressures for CO and H₂ respectively). So, the factor of 12 to 15 increase in ρ_{CO} needed for mid-alcohols may be offset by a factor of 4 increase in ρ_{H2}.

Note that the transient conditions at start-up or shut down are radically different from the steady-state conditions (described in this discussion) because of the need to build up considerable excess CO in the FTS reactor relative to the stoichiometric ratios, but that can be ignored in the steady-state analysis with efficient CO recycling. Also, additional FTS temperature control via some waste heat rejection to the environment, as well as some electrical heating, may be required during transient conditions. Further advances in reactors for high-pressure, variable-rate, low-conversion, high-temperature, exothermic reactions will be disclosed separately.

Initial Separations and Enthalpy Recovery. The composite vapor product from the HT FTS reactor goes through a series of partial condensers at sequentially lower temperatures but still at full pressure—partly to achieve more mid-grade heat recovery that can be utilized elsewhere. The primary object is to separate the gases (CO, CO₂, H₂, CH₄, C₂H₄, etc.) with minimal energy penalty. Another objective is to minimize utilization of the high-mid-grade FTS waste heat where low-mid-grade waste heat can be used so more of the higher-grade waste heat remains available to drive heat engines. The number of primary condensers shown is much higher than normally seen in FTS systems, but it is probably about right. The initial partial condensations take place essentially at the FTS pressure, though gas recompression losses may be tolerable with pressures as low as two-thirds or even one-half of the FTS pressure in some condensers. This is distinctly different from standard distillation processes with petroleum, where the initial distillation takes place at atmospheric or sub-atmospheric pressure with very minor non-hydrocarbon gas fractions.

The separations process chosen here is more related to that employed in some cryogenic air separations plants; and the primary governing equations are Henry's law on gas solubility and Raoult's law on partial vapor pressures, as also encountered in gas-liquid absorption processes. A primary objective is to achieve highest practical efficiency in recycling of the unreacted synthesis gases and at the same time achieve rough separations of the synthesized products with minimal waste.

As noted, high selectivity for alcohols requires low CO conversion per pass, which implies very high recycle of CO, H₂, and CO₂. It also means the gas leaving the FTS reactor will be quite “dry” or “lean”—that is, will require considerable cooling before any significant condensation occurs. Cooling the reactor vapor products to just above the temperature at which significant condensation begins, about 450 K, in a separate heat exchanger or regenerator 30 allows for more effective utilization of this substantial quantity of high-mid-grade heat.

The first three partial condensers 31, 32, 33 (420 K, 380 K, and 345 K) are cooled by pressurized water, and this enthalpy can be used in the syngas and feed-gas preheating. The temperatures indicated may seem surprisingly low for the high-pressure conditions, but the high gas fraction requires very low vapor partial pressures for condensation to occur. For reference purposes, the vapor pressures of some FTS products are noted in the various condenser boxes at their respective condenser temperatures for the pure substances. Some of the major condensed products and typical flow rates are noted in the boxes between the liquid output stream reference numbers, L1-L8, and their respective condensers. Keep in mind that the temperatures and other conditions mentioned are only representative of the suggested mean operating conditions. We later discuss how temperatures, pressures, and flow rates would change as available electrolyzer power changes. The enthalpy from the fourth condenser 34, still at near the FTS pressure, is rejected to the atmosphere at 310 K in a cooling tower.

Cryogenic Gas Separations and Recycling. While satisfactory short-term FTS performance might be possible with CO₂ molar fraction in the feed syngas above 25%, it will probably be necessary to keep the CO₂ molar feed fraction below 15% for acceptable catalyst lifetime and low production of organic acids. Keeping the feed CO₂ below 5% may be justified for optimum catalyst selectivity and lifetime. It may also be justified by the benefit it provides in separation of inerts and reduction of regenerator costs, as discussed later. The best method here for getting enough CO₂ out of the recycled syngas for efficient FTS operation includes cryogenic separation, which is quite compact and efficient at high pressures and permits simplified separation of more of the very light products.

Pressure swing absorption (PSA) of CO₂ using monoethanol-amine (MEA) has more often been used for separation of CO₂, but it (A) has rather high energy requirements for solvent regeneration, (B) requires considerable energy for CO₂ recompression, and (C) adds water and amines to the gas stream, which subsequently must be removed. A recent MEA CO₂ separation example for a complex stream required 6.2 MJ/kg-CO₂, which is an order of magnitude higher than can be achieved by a cryogenic separation method when the gases are already in a high-pressure loop.

The availability of a huge amount of pressurized oxygen from the electrolyzer presents a novel opportunity for what amounts to free refrigeration. (Both H₂ and O₂ are generated at high pressure, as that is required for high efficiency in the electrolyzer.) While the current market value of the oxygen is much greater than the value of its cooling power, the local oxygen markets may collapse by the time the third 250 MW wind-fuel plant is built in any region. It would then be better to use most of the available waste oxygen for other purposes, including refrigeration for cryogenic separations. Even more cooling capacity is achieved at very low cost from insertion of a compressor and expander into the novel primary recycle loop, as seen in the following.

In a gas stream in which the sum of the molar fractions of the light gases (H₂+CO+CH₄+C₂H₄, etc.) is about 80%, the CO₂ molar fraction cannot easily be reduced below 15% by cryogenic separations when the total gas pressure is only 8 MPa—because the vapor pressure of pure CO₂ just a few degrees above its freezing point is about 1 MPa. To achieve more complete CO₂ separation requires a higher condenser total pressure than would be optimum in the FTS reactor, especially when the FTS reactor is operating at low-power conditions. Hence, the recycled stream must be compressed in compressor 35 to the pressure needed to achieve sufficiently effective CO₂ separation by cryogenic methods (the pressure needed is dependent on the light gas fractions). The best place to insert this extra compression would be after the 310-K condenser 34. The gas mixture under the conditions here has relatively high C_{P} and C_{P}/C_{V} ratio, or “γ”, so the compression would initially appear to be quite costly from an efficiency perspective. However, much of the compression power required here will later be recovered in an expander, as will be seen, and that expansion is also needed for cooling. Assuming 8.6 MPa in first ambient-temperature FTS condenser 34, a pressure ratio of about 1.4 is needed to get the 12 MPa total pressure needed to achieve a CO₂ molar fraction below ˜15% from condenser 39. However, in some cases, cryogenic condenser pressures as little as 10% above the FTS reactor pressure may be sufficient for adequate CO₂ separation.

Because of the desire to minimize separation penalties and purging, the equilibrium level of total inerts (CH₄, Ar, N₂, He, etc.) in the FTS loop is higher than might be expected. A typical gas mixture for the compressor might be: 12.5 kg/s CO, 0.7 kg/s H₂, 7 kg/s CO₂, 1 kg/s N₂+Ar+He, 1.3 kg/s CH₄, and 0.8 kg/s other light HCs and alcohols. The mean molecular mass (m.m.) is about 21, mean gas C_{P}=1.6 kJ/kg-K, and γ=1.47. Assuming polytropic compressor efficiency of 88%, the compressor probably requires about 1.5 MW and the product gas comes out at about 355 K. (There is considerable scatter from published models for such a mixture at these conditions.) Thus, the next step is a second ambient-temperature FTS condenser, 36. This condenser 36 produces a very small liquid-product stream L5 of mostly light HCs, but it is essential for maximum efficiency.

The first cooled FTS condenser 37 operates just above the water freezing temperature for maximum water removal prior to the cryogenic condensers—though probably over 99% of the water has already condensed out, owing to its high solubility in the mid-alcohols and other oxygenates. Because of the high solubility of the light HCs and CO₂ in the middle HCs, a significant amount of the former condenses here into L6, in spite of the high gas fraction. Subsequent partial flashing of these gases can provide a small fraction of the cooling needed here, as shown in FIG. 3, but most must come from other sources. Most of the cooling can be provided by the cold recycled syngas, and some may be provided by an oxygen cryocooler. Both will be discussed shortly.

The second cooled FTS condenser 38 operates about mid-way between the freezing points of water and CO₂. At 12 MPa with a high CO₂ fraction, a majority of the liquid stream L7 may be CO₂, and it acts as a solvent in pulling more of the residual light HCs out of the non-condensable products. Subsequent staged flashing of L7 can provide a significant fraction of the cooling needed here, but still most must come from other sources. Again, most of the cooling can be provided by the cold recycled syngas, and some may be provided by an oxygen cryocooler.

The final condenser 39 is as cold as is practical without freezing the CO₂. Here, boiling the condensate 45 may provide much of the needed cooling, and the cold recycled syngas also provides some, as shown. However, cooling at the lowest end of the cooling range must be provided by a colder stream, and this may be an oxygen cryocooler. For the conditions in this example, only a very small fraction of the CO₂ can be condensed and the rest continues on with the cold recycled high-pressure (HP) syngas to regenerator 40. The condensed CO₂ helps wash some very light HCs from the remaining gases. Without the excess CO₂ from the WGS in the FTS reactor, a smaller fraction of the valuable light HCs would condense out here. Of course, they can be even more effectively recovered downstream of the final condenser in an oil absorption column, as discussed later in the context of CH₄ separation.

With highly effective regenerators or recuperators (and practical losses), heating the HP syngas to 270 K can provide over 1.5 MW of the cooling power needed in condensers 38 or 39. It is important to appreciate the significance of using regenerators or recuperators for these heat transfers, as opposed to using phase-change fluids, which have been proposed by Severinsky and others. It would be quite complex to recover more than half of the available cooling power from the cold syngas using phase-change fluids.

The HP syngas leaving regenerator 40 at about 12 MPa and about 270 K is then expanded in turbine expander 41 to essentially the FTS reactor input pressure, 9 MPa. Assuming an expander polytropic efficiency of 85% and γ about 1.64, the syngas temperature drops to 225 K and about 1.5 MW_{E} of electrical power is generated. (Again, there is considerable scatter in published models here.) By using highly effective recuperators, over 2 MW_{T} of additional cooling power is now available for condensers 37, 38, and 39 from the process of heating this cold syngas to 290 K. In this case, advanced recuperators, as disclosed in a co-pending patent application, are needed, as there is a substantial pressure difference between the streams.

There will be enough ethylene, propylene, and butenes remaining in the syngas after the final condenser 39 to be worth further separation for sale, and inert gas separation is also needed. Some possibilities for such separations 42 are discussed under Other Main-process Gas Separations.

The recycled syngas/hydrocarbon mixture is then heated to the extent practical in heater 44, preferably using a sequence of low-grade, mid-grade, and high-mid-grade waste heat sources. The total amount of heat needed here is fairly large (about 9 MW total); but there is no shortage of low-grade heat available, and the needed higher-grade heat could be transferred from 30, possibly using a regenerator, as the pressure difference between 30 and 44 is quite small. Minor amounts of other vaporized byproducts, such as methanol and acetone, could also be injected directly into this re-heated syngas if their markets are weak and a better reformation process is not justified.

Additional Comments on the Primary Loop. All the condensed, high-pressure output streams, L1-L8, will contain substantial amounts of both lower boiling and higher boiling species. Their lower-boiling fractions will largely come out in staged depressurization (flashing), which is not shown here. There is no significant HC venting or purge stream, and essentially everything is sold or recycled.

Some liquid CO₂ will come out in L6 before the first cryogenic (250 K) condenser 38, but some CO₂ will also be in L5, L4, L3, and even L2. Most of this can be separated and recycled 49 cost effectively into the RWGS CO₂ feed 19, perhaps after separation of ethylene 46, 48 and ethane. A fraction of the CO₂ may be available at sufficient pressure for injection at 12 ahead of the CO₂ heat engine. Of course, it may also make sense to vent some of the very minor CO₂ streams that evolve from liquids at low pressures.

A preliminary simulation shows the sum of the molar fractions of the methanol, acetaldehyde, ethanol, acetone, and propanols in the vapor stream leaving the 280 K condenser 37 will be an order of magnitude greater than the water molar fraction (which will probably be less than 0.03%), so it will be possible to achieve long runs between defrost cycles. It is useful to note that, with cryogenic separations, icing problems would be much greater for FTS products other than alcohols, as (A) the high ratio of alcohols to water in the cryogenic condensers keeps the residual water there from freezing, (B) the alcohols increase water removal in the higher-temperature condensers, and (C) less water is produced in alcohols synthesis. The ambient-temperature condensers 34, 36 for liquid streams L4, L5 may need to be water/ethylene-glycol cooled (as their temperatures may drop below the water freezing point during calms on cold nights).—

Again, the high-pressure, multi-level, partial condensation process shown is quite different from the fractional distillation processes used in the petrochemical and fermentation industries, which are normally carried out below 200 kPa and sometimes even below 10 kPa. It also differs greatly from prior FTS separations processes, where the FTS product usually contains a much lower gas fraction (so low-pressure separation processes more similar to conventional petrochemical multi-cut distillations can be used with good efficiency).

An Oxygen Cryocooler and Heat Engine. FIG. 6 illustrates a method of using the source oxygen to generate cryogenic cooling in a partial, open, reverse Brayton cycle. The example electrolyzer provides 11 kg/s of source O₂ at 4 MPa, 430 K, as shown at 81. Here we assume one-third of the wet O₂ is desired for the cryocooler, but before use in a cryocooler this wet oxygen needs to be dried.

Most of the water condenses out at 83 upon cooling to 300 K in 82 (designated RT-O₂), which requires 1.3 MW_{T} for 4 kg/s of wet O₂. Refrigeration in condenser 84 to about 280 K (using some of the excess cooling capacity from 42, for example) reduces the water content well below 0.1%. Further drying would also normally be desired (perhaps by absorption in triethylene glycol, TEG, C₆H₁₄O₄). Expanding this oxygen by a pressure ratio of 8 through a turbine 85 with 84% efficiency generates 400 kW of electrical power, and the oxygen emerges at 160 K. This first cold oxygen stream 86 can provide nearly 0.2 MW of cooling to the coldest FTS cryogenic condenser (39) and emerge at 220 K before needing to be expanded again.

Expanding this cold O₂ in turbine 87 by a pressure ratio of 4 generates 150 kW of electrical power, and the gas emerges below 160 K, which can again provide nearly 0.2 MW of cooling 88 to the 225 K condenser 39 in the RFTS plant. Further cooling is available at increasing temperatures in exchangers 89, 90 for partial cooling of condensers 38, 37 and 84. While there is ample cooling available from this final expansion for that needed in the 280-K condenser 84, it is more efficient to use the lower temperature cooling, especially from exchanger 88, for lower-temperature needs, such as in 38 and 39, and use some of the cooling capacity from a higher temperature stream such as 42 to supply the balance of the cooling needed in 84. Some of the vented dry oxygen 91 may be useful in a CPOX reaction, discussed later.

The balance of the 4 MPa oxygen, here assumed to be 8 kg/s, may be used in a heat engine if there is insufficient market to justify liquefying it for sale. A partial open Brayton cycle is the best option. First, low-mid-grade heat 92 and then high-mid-grade heat 93 are used to heat the oxygen to 590 K before expanding in turbine 94 to 0.7 MPa, 390 K, and generating 1.5 MW of electrical power. The turbines and heat exchangers with sufficient oxidation resistance will be more expensive than for the CO₂ heat engine, but the requirements are not extreme, as it has a turbine inlet temperature of only ˜600 K. Note that the amount of electrical power generated here is nearly twice the amount of high-mid-grade heat needed. This is possible because of the mechanical energy in the high-pressure gas produced by the electrolyzer. This energy is usually ignored in standard HHV definitions of electrolyzer efficiency. Clearly, this is a very good way to use FTS waste heat.

The expansion ratio chosen in expander 94 is somewhat arbitrary, but one cannot expand this pressurized oxygen all the way to atmospheric pressure without some inter-heat, or ice would form in the final expansion turbine, leading to blade erosion. The amount of inter-heat 95 is also somewhat arbitrary. Here, we have chosen not to use any high-mid-grade (FTS) waste heat to show that an additional 1.3 MW_{E} can be generated by expansion 96 to atmosphere from only 0.9 MW_{T} of additional lower-grade heat. The waste oxygen exhausts (with no H₂O condensation) at ˜330 K.

Other Main-process Gas Separations. While most of the FTS product initial separations are from partial condensations, some additional gas separations are required in the main loop that are not detailed in FIG. 3, as they are of relatively little overall significance. Several additional separations include: some separation of CO₂ (for recycling back into the RWGS) from the RWGS product; separation of CH₄ from the recycled syngas; and separation of trace amounts of other inert gases. Of course, there will be many separations associated with the product streams, some of which were mentioned earlier and some are discussed later.

To keep the CO₂ in the FTS reactor 29 low enough for the desired level of performance, the CO₂ coming out of an RWGS reactor system as presented in FIG. 4 will probably need to be separated 24 and recycled 20, as shown. Cryogenic methods, as used at the end of the FTS output stream, are not effective in achieving low CO₂ molar fraction at the relatively low pressure seen here.

Potassium carbonate (K₂CO₃) in hot water is often used in newer ammonia plants to separate CO₂ from H₂ and N₂, though usually at higher pressures. It is inexpensive and very efficient when plenty of waste heat is available. Moreover, it appears an extremely efficient electrolytic method of solvent rejuvenation, as disclosed recently by Martin and Kubic, may soon be available. However, PSA using an amine of high stability and low volatility may be preferred. The CO₂ separation process might be inserted prior to rather than after the drying step 22. Drying is also required in both exit streams from the separator 24. A simplification here compared to standard commercial applications is that neither high recovery nor high purity is required in this separation.—

Keeping the level of CO₂, CH₄ and other inerts low in the recycled FTS process reduces the size and cost of all the components in the FTS recycle loop. Without explicit inert-gas separation, its equilibrium level (in the FTS recycled syngas) might be 20-50%, as determined by the rates at which the gases are added and removed by other processes. The rate of CH₄ removal must normally be much greater than that of the other inerts, as it is usually the one being added (from new syngas and FTS processes) at the highest rate. The primary inerts are normally removed from the loop at roughly similar rates by the other product condensations in streams L3-L8. All of the inerts are of similar consequence within the FTS loop, and keeping their sum in the 4-15% range is normally justified. While CO₂ is not inert in the FTS reactor, its detrimental affect in the FTS loop is greater than that of the inerts, as it also increases acid formation in the FTS reactor and thus decreases its lifetime.

Both membranes and solid adsorbents have been used successfully for CH₄ separations, but usually from only one or two of the main syngas constituents at a time. For the past three decades, gas separation by chilled oil absorption has usually been considered archaic, but it appears to be the best option for reducing CH₄ to 5-10% molar fraction in this product stream (˜45% CO, ˜35% H₂, ˜12% CO₂). A standard absorption column, flash drum, and possibly a stripper are required, somewhat similar to that shown for the CO separation process in FIG. 5.

At high pressures near ambient temperature, the solubility (reciprocal Henrys) of methane in chilled, light oils is typically over twice that of CO and less than half that of CO₂, but it is often two orders of magnitude greater than that of hydrogen. Clearly, a lot of CO₂ will be removed along with the CH₄ in an oil absorption column—and this would allow the use of a lower pressure in the cryogenic condensers. The solubilities of all HCs above C₁ in oils are much higher than that of methane, so one argument for separation by oil absorption is that it simplifies recovery of residual, light, valuable HCs from the recycled syngas and it separates the other inerts (Ar, N₂, and He) fast enough. Moreover, it allows the CO₂ content in the recycled syngas to be easily reduced at little additional cost (which may improve catalyst lifetime and product mix). The CH₄ separation 42 is shown in FIG. 3 after the expansion to the FTS pressure. Placing the CH₄ separations before expander 41 may be more efficient, as the higher absorption pressure allows lower recompression losses, though CH₄ selectivity relative to CO₂ may be better at lower pressures.

The solubility of CH₄ and the ratio of solubilities of CH₄ to CO plus CO₂ are the most important parameters in oil selection, though there is enormous scatter in such data for light oils, especially below 380 K. Too much removal of CO, H₂, and even CO₂ is detrimental to efficiency, as they require subsequent separations and compression. Methane separation using cold octane, perhaps at ˜240 K, appears to be a satisfactory option. The absorber vapor added by this column can easily be removed from the methane-depleted syngas in a second oil absorption column using a heavier oil. Elsewhere it will be shown that the CH₄ can be separated at a total cost of under 8 MJ per kg of CH₄ removed while the other residual HCs are also recovered at very low cost.

There are many viable options for the separations of the off-gas from the above methane separation, as both the flow rate and H₂ fraction there are relatively low. The best approach is probably to begin by removing the CO₂ by amine absorption, followed by a second cold-oil absorption column to separate the higher HCs (C₂H₄, C₂H₆, C₃H₆, etc.) from the gases (CH₄, CO, H₂, Ar, and N₂). This gas mixture (mostly CH₄ and CO) can then be separated efficiently using membranes and solid adsorbents. The higher HCs would be send to product separations. The CO and H₂ are sent to the input of the new syngas compressor 26, and the CH₄ can be burned or reformed to syngas.

If the recycle-RWGS process as shown in FIG. 5 is used, inerts will build to a rather high equilibrium level within the RWGS loop—a level that is largely determined by the ratios of their solubilities and K-values in the aromatic for the conditions in the stripper and absorber and by their concentrations in the source gases. Most likely, the CO separation process, such as COSORB, would keep the total inerts in the RWGS loop below 12%. The biggest challenge is the CH₄, as noted earlier. If it is excessive, the easiest way to deal with it may be a small purge stream from point 426 to another separation process. Membranes could be used to recover the valuable H₂ and HCs from the purge stream after separation of the CO₂ by amine absorption. A small purge stream at 43 with subsequent similar separations may be used to further limit the argon, N₂, and He in the FTS loop, though they should be removed at a sufficient rate by the CH₄ separation process.

In summary here, it seems safe to assume that the net efficiency penalty for the various primary gas separations that are not well detailed in FIG. 3 can be under 1.5% and their costs will also be minor.

Secondary Separations and Upgrading. The amount of CO₂, CO, H₂, CH₄, C₂H₄, C₂H₆, C₃H₆, C₃H₈, and C₄H₈ dissolved in some of the pressurized liquid streams L1-L8 (especially in streams L1-L3) may seem too small to merit recovery (as their sum, excepting CO₂, is normally in the range of 1 to 10% there). However, it will be very easy to achieve efficient separation of the very lights from the higher-boiling components, as the necessary hardware will be present for other purposes. After flashing these liquid streams, the flash-gas mixtures of CO₂, CO, H₂, CH₄, and other lights could simply be sent through a small condenser (˜250 K) and then to the processing of the off-gas from the cold-oil used for CH₄ separation discussed earlier. The light olefins are particularly valuable feedstocks for plastics, reagents, and chemicals of all types, so the C₂H₄, C₃H₆, and C₄H₈ would be separated (possibly using membranes or solid adsorbents) and sold.

Some secondary off-gas separations could utilize other methods. For example, PSA using a K-promoted hydrotalcite (K-HTLc) has good performance for CO₂ separation at flue-gas conditions. Better results for CO₂ adsorption near ambient conditions have been obtained with molecular sieve 13X (0.8 nm), where excellent preferential CO₂ adsorption is obtained in the presence of N₂, O₂, H₂, and H₂O; but there is insufficient data in the presence of large CO content. There is reason to believe the preference ratio relative to CO would not be adequate in most cases. NaY zeolite has order-of-magnitude preferential absorption of CO₂ relative to CO at 0.1 MPa in the binary gas mixture, and extrapolations to 1 MPa suggest good selectivity there too, but data for the tertiary mixture are rare. Zeolite (molecular sieve) 5A (0.5 nm) is often used for CO, CH₄, and N₂ separations. Another possibility for the separation of the CO₂ from H₂ and CO is a reverse selective membrane, as disclosed in U.S. Pat. No. 6,572,680. Since CO₂ is highly soluble in some rubbery membranes (available from Membrane Technology and Research, Menlo Park, Calif.), they can exhibit CO₂/H₂ selectivities above 10.—

There will be use for a small fraction of the O₂ (some of that vented at atmospheric pressure from 91 or 96) in reforming of excess low-value products—especially CH₄—via catalytic partial oxidation (CPOX) into new syngas—which will be much more valuable than CH₄ for at least the next 15 years. Some exothermic CPOX reactions may be carried out above the RWGS reactor temperature so their reaction heat may be used to assist the RWGS reaction. The best use of C₂H₆, C₃H₈, and C₄H₁₀ may be dehydrogenation to their respective (much more valuable) alkenes.

The less volatile remainders of the crude liquid streams will go through additional distillations and upgrade processes (drying, hydroisomerization, etc.) that result in the desired liquid product streams. Powerful, low-cost software, such as Design II, from www.WinSim.com, has been available for more than a decade that makes it easy to design many efficient separation processes—except membrane and solid-adsorbent separations. Distribution of liquids to global markets is quite efficient.

The heavy products from a mid-alcohols plant constitute a very small fraction of the FTS product, but this small stream of wax from the FTS reactor (as well as a little soft wax condensed from L1) needs to be efficiently upgraded. As disclosed in U.S. Pat. No. 6,939,999, it can be beneficial to catalytically dehydrate the undesired oxygenates to their corresponding olefins prior to hydrocracking. These heavy products would then be hydrocracked to naphtha, diesel, and jet fuel by adding high-pressure hydrogen in the presence of the right catalysts; usually 25-40 kg H₂/ton heavy feed is sufficient. The resultant hydrocracker liquid product flow rate typically has 5% greater energy density than the feed liquid and 20% higher flow rate. Some additional hydrogen may be desired for hydroisomerization, production of high-value chemicals such as cyclohexane, or for sale in the local hydrogen market. Therefore, an additional 10 MW_{E} electrolysis power is assumed needed here to generate hydrogen for these purposes.

Wax (n-C₃₀-C₁₂₀) that is free of sulfur, halides, metals, and nitrogen can be efficiently hydrocracked and isomerized to jet fuel (C₉-C₁₆), diesel (C₉-C₂₅), naphtha (C₆-C₁₅), and high-value lubricants. (The upgrading catalysts are often even more sensitive to poisons than the FTS catalysts.) Fluidized catalytic cracking (FCC) and hydrocracking may be used to convert lower-value longer chain molecules to higher-value, shorter-chain molecules. Upgrading of the lower value byproducts will be much simpler in the wind-fuel plant than has been the case in previous GTL plants—partly because of the ready availability of high-purity hydrogen and essentially free high-purity oxygen and cryogenic cooling, even at the smallest WindFuels plant.

Up to 10% of the carbon might be emitted as CO₂ from combustion of low-value byproducts to drive the multi-stage RWGS, but these CO₂ and H₂O combustion products would more likely be recovered and separated for use as inputs to the RFTS plant. Since high-purity O₂ is available at very low cost for this combustion, recovery of the combustion products is quite simple. There will be small mixed streams (including acetates, glycols, acetic acid, phenol, and possibly tars) of products from secondary separations that are not produced in sufficient quantity for on-site upgrading or purification. These would go to a regional chemical processing plant for upgrade and utilization. The high-pressure cryogenic separation process makes it easier to separate the various FTS products which have n.b.p. below ˜320 K from the heavier hydrocarbons and generally prevent the venting or loss to the atmosphere of any significant amount of these byproducts. Of course, some of the gases and vapors from the various condensed streams may evolve in complex mixtures too small to be worth separating and upgrading. Normally, there is no significant purge stream, and the total carbon loss rate would preferably be under 1%. However, in very small RFTS plants, up to 30% of the very lights might be vented. The water separated from streams L1-L6 may need a fairly complex purification process, but effective processes for such have been disclosed by Kohler et al in U.S. Pat. No. 7,166,219.

RFTS Plant Efficiency. Calculation of the FTS reactor heat production based on that released from the earlier assumed FTS selectivities, using calculated 600-K reaction heats (˜28 MW_{T}) plus the assumed WGS activity (˜3.5 MW_{T}) suggests the FTS reactors will be generating ˜31.5 MW of reaction heat. (Most of the synthesis heats are about 10% higher at 600 K than at 298 K.) If the HT-FTS reactor output temperature is 60 K above the temperature of its inputs, all but 3 MW of this heat would be available for export (except for perhaps 1.5 MW of reactor losses). Note that this reaction heat is much less than might be expected based on other FTS experience because the 600-K FTS-reaction heats for methanol (3.14 MJ/kg), ethanol (5.91 MJ/kg), propanols (7.21 MJ/kg), and even propylene (9.41 MJ/kg) are much less than those for most other products (for example, 11.5 MJ/kg for the mid-cut alkanes). The total HHV of the products, which total 4.4 kg/s (including the CH₄ from the RWGS reactors passing through the FTS reactor) amounts to 152 MW for an assumed 1.35 kg/s of H₂ going into the RWGS and FTS reactors. From the assumed selectivities, some outputs would be: 1.1 kg/s ethanol, 1.1 kg/s methanol, 0.35 kg/s propanols, and 0.17 kg/s propylene.

It is useful to look at plant input and output thermal tallies for the near-term RWGS case, that shown in FIG. 4 and assumed in FIG. 3. On the thermal-out side, after syngas heating in the FTS reactor and its losses, we have: 27 MW of high-mid-grade heat (560-630 K, 29); 8 MW of low-mid-grade heat (460-560 K, 7 MW from 30, and 1 MW from 26 intercool); 8 MW of low-grade heat (360-460 K, 1 MW from 30, 2 MW from 26 intercool, 5 MW from 31, 32, 6, and 50); and 36 MW of near ambient heat rejection (300-360 K), mostly from the condensation of water (from the RWGS reactor and from the wet source O₂ and H₂). There is also about 1 MW of heat in the higher condensed streams after partial flashing, some of which is low-mid-grade (the wax and L1), but most is low grade. Finally, about 0.5 MW leaves through supplemental chillers. That shown specifically in FIG. 3 as transferred is not counted on either the plus or the minus tally. Hence, the thermal-out sub-total is about 86 MW.

On the thermal-in side there is: 15 MW for the H₂ heating; 3 MW for the CO₂ heating; 13 MW for the RWGS heating; 12 MW for the syngas preheating; net electrical input is about 6 MW_{E} (about 1 MW_{E} is not shown); about 3 MW is needed to account for the source hydrogen being at 430 K rather than 300 K; and the mechanical and latent energy in the (compressed, wet) source hydrogen amount to about 6 MW. This gives an input subtotal of about 58 MW in addition to the 190 MW of hydrogen HHV chemical energy at 300 K.—

How effectively the lower grades of heat are utilized doesn't effect the FTS reactor heat generation, but it does affect how much of the FTS heat remains available to drive heat engines. The final H₂ heating (about 2 MW of that in 9) and all of the RWGS reaction heating (13 MW into 10) may need to come initially from a high grade source—either resistive heating or combustion of low-value byproducts. Some of that for the source-gas heat engines (2, 4, 12, 94) and H₂ mid-heating 9 (a subtotal of about 8 MW for these purposes) will need to come from the high-mid-grade heat source, the HT-FTS reactors 29. The additional lower-grade heats needed (9 MW for H₂, 2 MW for CO₂, 1 MW for O₂, 10 MW for syngas) are mostly supplied by the previously mentioned low and low-mid grade rejections, though some additional is needed from the electrolyzer rejection (430 K). The non-cryogenic separations 24, 42 will also require some low-grade heat from the electrolyzer. Most of the 36 MW rejected just above ambient temperature will not be of value, though some can be used in vacuum distillations in product upgrading.

In this example, we assumed about one-third of the O₂ will be used for supplemental cooling and the balance will be used in a heat engine, which requires only 0.8 MW of high-mid-grade heat 93 and 1.5 MW of low-mid-grade heat 92, 95. This generates over 0.3 MW_{T} of sub-220-K cooling and about 3.4 MW_{E} of electrical power. The actual amount of cooling assist needed in the FTS plant will depend greatly on the effectiveness of the regenerators and recuperators (40, 43, etc.) and on the pressure ratio in the FTS-loop compressor 35.

Prior to the byproducts upgrading, the FTS plant of FIG. 3 with the above O₂ section has about 2.5 MW net electrical input power requirement. Reclaiming and recycling the various flash gases, together with other separations and upgrading operations, are likely to consume about 2.5 MW_{E}, bringing the net electrical power requirement (for FTS plus oxygen plus upgrading sections) to about 5 MW_{E}.

There is still (after driving the source-gas heat engines) about 19 MW_{T} of surplus high-mid-grade waste heat available to drive another heat engine. Steam turbines with only a 590 K source temperature (20 K below the FTS reactor) would normally achieve about 30% efficiency. (For reference, the typical thermal efficiency in CSP plants where peak fluid temperatures are about 640 K is currently about 32%.) However, the availability in the RFTS plant of abundant low-grade heat (˜50 MW_{T} at perhaps 430 K) from the electrolyzer will allow utilization of the high-grade heat at much higher efficiency. A co-pending application discloses a novel Dual-source doubly-recuperated Organic Rankine Cycle (DORC) that achieves much higher efficiency while simultaneously reducing the cost of the heat engine when both a low-grade and a mid-grade heat source of comparable magnitudes are available.

In this RFTS case, it appears that a DORC driven by 19 MW_{T} at 600 K and 15 MW_{T} at 420 K could generate over 10 MW_{E} output. After providing the 5 MW_{E} of needed electrical power noted earlier, about 5 MW_{E} is then available for other purposes—presumably, more electrolyzing.

About 8 MW worth of additional chemical power (0.056 kg/s of H₂) was allocated earlier for hydrocracking, upgrading, and local hydrogen sales. About half of that used in exothermic upgrading adds to the chemical power of the products, and about half is rejected as mid-grade heat.

Recall that the total FTS output product stream is 152 MW HHV, and the extra hydrogen stream adds about 6 MW. About 13 MW worth of low-value products was needed to drive the RWGS. The 5 MW_{E} of surplus electrical power is most easily dealt with by subtracting it from the 250 MW of assumed source power. Hence, net plant efficiency, including the electrolyzer with the RWGS method of FIG. 4, appears to be 144/245, or over 59%. The RWGS method of FIG. 5 appears to achieve slightly higher efficiency and ultimately lower equipment cost. This method may ultimately allow the RWGS reaction to be driven by FTS waste heat and achieve up to 4% higher efficiency.

Depending on the needs for some of the CO₂ separations, there may still be about 20 MW_{T} of low-grade (430-K) heat available (above that needed for the DORC) from the electrolyzer that could be converted to additional electrical power or used for steam heating of local businesses and residences. Most of the heat rejections at lower temperatures would not be usable.

It is possible that it will not yet (until energy becomes even more valuable) be cost effective to add the DORC, especially if there is a better use for most of the electrolyzer waste heat. If this or a similar heat engine is not included, much more higher-grade waste heat would be available. Using this higher-grade heat where lower-grade heat would be adequate would allow a substantial reduction in the cost of the various heat exchangers.

Miscellaneous Efficiency Considerations. The mid-term electrolyzer efficiency assumed above, 80%, may be about 5% higher than the best current commercial technology by standard HHV definitions, but it is well below what has been demonstrated on research systems. The standard definition ignores the mechanical and thermal energy in the warm, compressed O₂ and H₂ gas streams. The 15 kg/s electrolyzer water is assumed supplied pre-heated to ˜430 K by about 8 MW_{T} of low-grade waste heat. The thermal energy in the source gases relative to 300 K is about 4.4 MW_{T}. Ideal isentropic expansion of both wet electrolyzer gases from 430 K and 4 MPa to atmospheric pressure (and about 150 K) would generate nearly 10 MW_{E}. If the mechanical and thermal energies are included in the source stream energy, the calculated FTS plant efficiency is about 5% lower (depending on definitions), but electrolyzer efficiencies are higher by a compensating amount.

Now that the mechanical and thermal energies in the electrolyzer outputs can be well utilized, it is more prudent to look at electrolyzer total (not HHV) efficiencies. The electrolyzer total efficiency can be increased considerably by dramatically increasing the pressure, as that greatly reduces bubble size and hence resistive losses in the electrolyte. A mid-term goal would be 15 MPa (where common elastomeric seals still work well for both O₂ and H₂), and a longer-term goal would be 70 MPa—the highest pressure normally seen in H₂ storage systems. The electrolyte resistivity is also reduced by operating with higher KOH concentration at higher temperature, though operation above 520 K, an upper practical limit for elastomeric seals, seems unlikely. Efficient handling of both product gases from variable-rate electrolysis at very-high-pressure (VHP), along with the conversion of the electrolysis heat that is now practical using the DORC, will allow higher cost effectiveness in renewable electrolysis than any other known electrolysis method. Note that the H₂O molar fractions in the wet electrolysis gases in the expanders may range from 0.5% to 50%, corresponding, for example, to electrolysis conditions of (A) 70 MPa, 412 K and (B) 2 MPa, 452 K, respectively.

Adequate allowances appear to be included for all major efficiency losses except for low-grade heats. Some of the low-temperature heatings were not handled in detail because there should be a surplus of low-grade heat if the minor separations are handled optimally. To demonstrate: about 8 MW_{T} of low-grade heat is required to heat the source water for the electrolyzer and even more may be needed for the CO₂ separators 24, 445, the CH₄ separator 42, and the CO stripper 442. The net amount of heat needed in the CO₂ separations is not too large, though they may (at least initially) use a substantial fraction of the electrolyzer heat and then reject much of it at a temperature too low to be of much use elsewhere. A 10 MW_{T} error on the low-grade heat needs and leaks would have negligible effect on net plant efficiency, though a 2 MW_{T} error on the high-mid-grade heat leaks would have a 0.7% effect on net plant efficiency when a DORC is included.

It may be perceived by those experienced in related processes, such as methanol production, that inadequate allowances have been made for pressure losses in all the heat exchangers. Indeed, that would be true if conventional gas-to-gas heat exchangers were used. Some unconventional designs with performance advantages for some of the conditions were discussed, and a highly advanced recuperator design is the subject of a co-pending patent application. However, an error of 1 MPa on total pressure losses of the FTS products through the fractional condensations would have only a 0.1% effect on net plant efficiency.

Perhaps optimistically low RWGS reactor temperatures were assumed. However, this has no significant effect on the amount of heat needed there when high-effectiveness recuperators are being used, though it means driving the RWGS reaction with FTS waste heat is more challenging.

If electrical power requirements for the non-cryogenic gas separations—which were not treated in detail—were underestimated by 30%, net plant efficiency would be about 0.3% lower. Adiabatic depressurization of a significant amount of gaseous FTS byproducts (such as CH₄ and C₂H₄) followed by recompression could reduce plant efficiency by up to 0.4%. Reasonable assumptions are used for heat exchanger temperature differences (mostly, 20 K to 70 K, though as little as 10 K where regenerators or novel recuperators are anticipated). Assumed turbine efficiencies, though higher than normally seen in GTL plants, are reasonable for the assumption that energy is much more valuable than assumed in historical designs.

Of course, many details could not be covered in a single document of acceptable length. For example, the temperature difference between the boiling of the CO₂ in 45 and that needed to condense it in 39 will probably be too small for practical heat transfer without a heat pump (this is an ideal application for such) with an appropriate working fluid, such as C₂H₆, C₂H₄, H₄Si, N₂O, CClF₃, CHF₃, or CH₃F. This may add 150 kW to the electrical load.

A significant variable is in the product upgrading, which depends on the details of the byproducts, the catalysts, and the upgrade product mix. The exothermic hydrocracking upgrading will probably generate all the high-mid-grade heat needed for the liquids separations. There is no shortage of low-grade heat available for reboilers in the various distillations and strippers, some of which may be at very low pressures, and some of which could be at very high pressures to facilitate the use of the available heat.

The best net efficiency measurement is the ratio of HHV chemical output power to electrical input power, which could be about 59% (here, ˜145/245) for mid-term performance. Some prefer to see LLV efficiency, and that number is ˜54%. Near-term performance may be lower by more than 4%, primarily because of current electrolyzer efficiencies. In practice, the objective will not be to maximize hydrocarbon energy, but rather to maximize profitability; so chemical output power undoubtedly would be less to allow the production of more chemicals of higher value or to reduce capital costs.

Off-design Performance. The above discussion has only addressed mean operating conditions in detail—240 MW_{E} from the renewable source plus ˜5 MW_{E} from heat engines. The winds are not steady, so accommodation of off-design performance is essential. The advanced wind farm is designed to be able to produce power during strong winds at nearly three times its expected mean level and not to stall at power levels as low as 10-25% of the mean power. The electrolyzer can respond very quickly to changes in available power as long as it is near design operating temperature and pressure. Its hydrogen and oxygen production rates are simply determined by the available current (to within less than 1%). Power fluctuations of very short duration (under 15 minutes) can be handled by charging and discharging a pressurized hydrogen gas storage facility—perhaps several, large, below-ground, steel tanks. Fluctuations of longer duration require adjusting the mass flow rates and hence the pressures and temperatures throughout the RFTS plant (and the number of FTS reactors in use). Some of the hydrogen storage would be as dried gas near ambient temperature at the RWGS entry pressure, and some would be at other conditions.

An important requirement will be adjusting the operating temperatures of the FTS reactors and condensers optimally so as to minimize variations in the product streams as the flow rate and pressures change. The FTS catalyst selectivity, the various chemical reactions, perhaps especially the degree of homologation, will change as a function of space velocity, temperature, and pressure, so the FTS product mix will change. However, it is not uncommon for reactors to operate over a rather wide range of conditions, and the product mix is not critical when the process is designed to efficiently handle a large number of products over a wide range of rates.

Standard turbines and compressors with fixed nozzles can often accommodate an order of magnitude decrease in mass flow rate without stalling and with only a 15-30% drop in efficiency if the pressure ratio drops by a factor of three and optimal changes are made in the rotational rates. Since the H₂ and CO₂ sources are likely coming in well above the RWGS pressure at mean conditions, higher expander and compressor efficiencies with fixed stator nozzles would be obtained as the flow rate increases by decreasing the RWGS pressure while increasing the FTS reactor pressure. In this way, the compressor and expander pressure ratios increase as the flow rate increases. However, much adjustment in this direction would lead to compromised RWGS performance at high flow rates, so variable-angle stator nozzles in the various compressors and expander turbines could improve performance over a wide range of conditions.

An alternative to variable-nozzle turbines for accommodation of the desired conditions is switching a number of fixed turbines in and out of parallel service. For example, if the expansion or compression power is expected to span the range of 1 to 7 MW, three turbines, optimized individually for 1, 2 and 4 MW, could be used in various parallel combinations to cover the full range more efficiently. Variable-speed motors, generators, and power conditioning permit efficient operation of standard turbines (compressors and expanders) over a wide range of speeds and pressure ratios if the speed is optimum for the pressure ratio, temperature, and mean molecular mass. However, the mass flow rate may be radically different than desired. The combination of switching turbines in and out of parallel service and allowing them to operating over a wide range of rotational rates can efficiently accommodate wide ranges in pressure ratios, mass flow rates, and temperatures. The electrical output from the expander generators would be used to electrolyze more water, so the conditioning of the variable-frequency, variable-voltage power from the various heat engines is simplified compared to most generator applications. Still, the development of variable-rate turbomachinery technology seems likely to be the most capital-intensive part of developing a variable-rate RFTS plant, as simple adaptations of standard aero-derivative compressors and expanders are not likely to be satisfactory for most of those needed in the RFTS plant. This development cost will likely mean that, for design and production economies, RFTS plants will be built only in several name-plate sizes, such as 30 MW_{E}, 60 MW_{E}, 125 MW_{E}, 250 MW_{E}, and 500 MW_{E}. However, each size plant could preferably operate very efficiently from less than half to more than twice its name-plate rating—possibly even for several weeks straight at peak capacity without slow down for reactor catalyst rejuvenations, cryogenic condenser defrostings, etc.

The operating pressure, volumetric flow rate, and temperature of the FTS reactor may be changed considerably in response to the desired mass flow rate with manageable changes in the makeup of the product streams. The mass flow rates in fixed-bed reactors can often be changed by a factor of two in either direction with acceptable changes in the FTS product mix if suitable adjustments are made in the reactor temperatures and pressures, the H₂/CO ratio, and the recycle ratio. Fluidized bed reactors, or the other hand, are much more difficult to fluidize properly over a wide range of conditions, and slurry reactors fall somewhere in between in flow-rate flexibility.

Having a high-performance compressor 35 and expander 41 (each able to efficiently handle the range of conditions) within the main recycle loop allows the reactor pressure to change over a wide range with considerably reduced effect on the cryogenic separations, which are beneficial in achieving the needed CO₂ removal and light-product recovery at high efficiency. Reduced flow rate would be accompanied by drops in reactor pressure and temperature and decreases in the temperatures of the higher condensers—and conversely for increases in flow rates. And as noted earlier, an even wider range in flow rates may be accommodated by having a number of parallel reactors that can be individually placed in or out of service.

The increase in residence time in the FTS reactor at low flow rates could largely be compensated by the reduced reactor temperature and pressure. The reaction rates as a function of temperature and pressure depend in rather complex ways on the micro-, meso-, and macro-structure of the catalysts, but it is not uncommon to see a factor-of-two change in many rates for a 30 K change in temperature at constant partial pressures. Adjustments in the H₂/CO/CO₂ ratios in the syngas would further help to maintain the desired FTS composite product mix.

It is important to appreciate that the efficient recycling scheme disclosed, along with independent control of the sources, makes it easy to obtain any desired H₂/CO ratio for any set of conditions without significant efficiency penalty—contrary to what is seen in non-renewable GTL. Control of the cryogenic condenser pressure and flexibility in the CO₂ removal from the final syngas in separator 42 make is much easier to achieve the desired CO₂ fraction.

The upper condensed-product stream compositions can be adequately controlled by adjusting the condenser temperatures 31, 32, 33 as needed. The various ambient-temperature condensers would generally be 3-15 K above either ambient or wet-bulb temperature, but also above freezing. The final drying condenser, 37, would usually stay just above the water freezing point for most effective moisture removal with minimal freeze-up problems.

It will be important to pay attention to the weather forecast so hydrogen reserves can be built to capacity in advance of a period of low winds, and so slow-down can begin early enough to maintain minimal reactor temperatures with available hydrogen reserves. If an extended lull is expected, it might be best to go into a standby mode where the temperatures are maintained at some minimal level with a single FTS reactor at nearly zero mass flow to simplify restarts.

It will be relatively easy to adjust the cooling powers for the cryogenic separations as needed to accommodate changes in flow rates; but the refrigeration—and in fact all the heat exchangers—must be sized primarily for peak flow rate, not the mean rate. The liquid product upgrading can be maintained at rather steady rates, as the raw liquids can easily be stored in large quantities during periods of peak production and upgraded at a steady rate during calms.

Efficiency during off-design operation will suffer less than might be expected. The biggest change will come from the electrolyzer. If the electrolyzer (and its power conditioning) losses are 20% (of line power) at mean power, they are likely to be over 35% at three times this power and 10% at one-tenth mean power. (The hydrogen production is very precisely proportional to the current, but the voltage drop increases at high currents.) The FTS methane percentage would increase with temperature and pressure and hence with the mass flow. The non-recovered losses associated with gas expansions and compressions are about 1.5% at mean design conditions (non-recovered losses are ˜16% of the sum of the absolute values of all the electrical powers to/from the compressors and expanders). Even with variable-rate turbines, these losses may increase to nearly 3% at both peak and minimum power.

The temperature differences in all the recuperators and regenerators (30, 40, 43, 62, 63 etc.) will decrease as flow rates decrease, and this will improve effectiveness (Nusselt numbers are nearly independent of conditions in high-performance gas-gas exchangers). Naturally, their effectiveness will decrease as flow rates increase above design mean.

The heat leak expected from the reactors, ducting, and high-temperature exchangers decreases only slightly as the flow rate drops. It becomes more significant at low power, but it will not be difficult to keep the total higher-grade heat leaks well under one-tenth of the mean FTS excess heat available. As the pressure ratios in the source-gas heat engines decrease at reduced flow rates, the amount of heat that can be effectively utilized there drops more rapidly than proportional to flow rate. From the combination of these effects, there should be sufficient waste heat to drive the source-gas heat engines adequately for mass flow rates below one-tenth of mean capacity. The amount of electrical power needed for the new-syngas compressor 26 at 10% of design flow rate may be only 5% that at design flow rate because its pressure ratio may be down by about a factor of two.

The net result is that, primarily because of changes in the electrolyzer efficiency, RFTS net plant efficiency will probably increase by 3-5% at half average wind speed (one-eighth mean wind power), and plant efficiency will drop by at least 12% during gales.

RFTS Design Variations. A mid-alcohols example was presented in detail because it appears to benefit the most from a high-recycle, high-pressure, cryogenic separation process. Given the current and expected commodities markets, it also appears to offer the most potential for return on investment. Thus, it would also offer the most potential for reduction in global CO₂ emissions. However, a similar cycle may also work well with a low-pressure, low-temperature FTS 3-phase slurry reactor for maximum diesel yield, as the cryogenic condenser pressure can be at much higher pressure than the FTS reactor. This might appear to require much higher FTS-recycle-loop compressor input power, and about one-third of that compressor power was not recovered in the subsequent recycled syngas expander in the mid-alcohols example. However, the diesel and gasoline FTS catalysts work well with much higher conversion per pass, so the ratio of the CO+H₂ recycle gas relative to the new syngas may be nearly an order of magnitude smaller. This will make it possible to achieve the desired CO₂ reduction in the recycle loop with lower pressure in the cryogenic condensers than in the mid-alcohols example.

Still, the CO₂ production in some LT-FTS slurry reactors is so low that it may be difficult to get significant CO₂ condensation in the final condenser without using very high pressure, and that would lead to a substantial efficiency penalty. Without much CO₂ condensation, there would be less condensation of the very light HCs into the liquid streams L6-L8, but they could still be efficiently separated by oil absorption at 42. A big challenge with slurry reactors would be in absolutely assuring 100% removal of catalyst fines ahead of the boost compressor 35.

It is not clear whether or not the high-recycle, high-pressure, variable-rate, cryogenic separation process would work well with the 2-phase fluidized bed reactors that have appeared to be optimum for maximum methane-based GTL-gasoline yield. However, the novel high-pressure process could certainly work well for high gasoline yield from an HT fixed-bed reactor, where the H₂/CO ratio would be closer to 2 and the pressure would likely be in the 1.5 to 4 MPa range.

The novel high-pressure cryogenic process would also work well for high yield of very light olefins using a fixed-bed HT-FTS reactor. For maximum ethylene yield, the reactor pressure may need to be below that preferred in the RWGS reactors, in which case the new syngas compressor 26 would not be needed. For high ethylene yield, the CO and H₂ recirculation would probably be high, so a lot of power would be consumed in the FTS-recycle-loop boost compressor, and an enormous amount of excess cryogenic cooling would be produced in subsequent, multiple, expanders following the final condenser 39. Some of this excess cooling capacity could be put to use in liquefying the waste O₂ for sale, and some would be needed for the RWGS method of FIG. 5. However, a more conventional approach to high yield of light olefins, in which the FTS reactor is optimized for naphtha which is subsequently cracked to light olefins, may be preferred.

The example presented in FIG. 3 showed the new syngas 27 being combined with the recycled syngas at 28 just prior to injection into the FTS reactor partly for conceptual reasons. It is not necessary that the two syngas streams be combined at that point in the main loop. In fact, it may be better to inject the new syngas immediately after the first ambient-temperature condenser 34 or after the recycle-loop boost compressor 35. Wherever the new syngas is injected, its pressure should be accurately matched to that at the injection point to minimize backstreaming into the upstream condenser.

The potential advantage of injecting the new syngas between 34 and 36 (rather than between 44 and 29) is that the CO₂ separator 24 can be eliminated. The gas flows through the cryogenic half of the recycle loop are then quite a bit higher than through the higher-temperature half, but the cryogenic condensers can be appropriately sized. The flows through regenerators or recuperators 40 and 43 remain adequately balanced for efficient cryocooling.

For the mid-loop-injection approach, the CO₂ separator 24 and RWGS-CO₂ recirculation 20 are eliminated. The dried RWGS product from the final RWGS condenser 68, still rich in CO₂, is compressed 26 to the same pressure as at the desired mid-loop injection point, adjusted to match the temperature at the desired injection point, and injected there. The extra CO₂ from this CO₂-rich new syngas appears as increased CO₂ in the subsequent condensed streams and still ends up back at the RWGS reactor as before. Which injection point achieves highest overall efficiency and lowest cost depends on many variables. A drawback of the mid-loop injection process is that a significantly larger amount of H₂, CO, and CO₂ must be handled by components 35 through 47—if injection was between 34 and 35, for example. Whether or not their increased costs exceed the cost of CO₂ separator 24 is not yet clear.

Herein, the liquid-stream separators have been called “partial condensers”, but they have also been known by other terms, including “fractionators”. This term sometimes implies that the exchanger for removing the heat is just upstream of the phase separator. The phase separator could also include a method for separating the polar from the non-polar condensate, in which case one or more of the liquid streams L1-L7 could emerge from its condenser as two separated liquid streams. A sequence of several partial condensers is also essentially equivalent to a multi-cut distillation column with cooled trays and without the reboiler or overhead condenser—though distillation columns seldom operate with such a large temperature difference between the top and bottom. Of course, a reboiler and partial overhead condenser could also be included, which may improve separations. However, it is easier to produce several partial condensers or fractionators than a high-pressure, wide-temperature-range, multi-cut, distillation column with heavily cooled trays. The latter would also have disadvantages with respect to maintenance.

The high-pressure cryogenic separation process appears to have advantages in high gas recycling and in flexibility for efficient recovery of a wide range of products from the FTS reactors. Moreover, the refrigeration capacity that comes from the utilization of pressure boost 35 and expansion 41 may be needed to efficiently implement the RWGS method of FIG. 5. (The use of a high-boiling solvent could obviate the need for cryogenic cooling in the solvent reclamations of FIG. 5, and other CO-separation methods that don't require significant cooling capacity might also be shown to be competitive with solution complexing methods.) However, the needed turbines 35, 41 make the high-pressure cryogenic process somewhat expensive, especially at smaller sizes.

Some of the above discussions have tacitly assumed the use of turbine compressors, some types of which are often referred to as centrifugal compressors. However, reciprocating, scroll, screw, sliding-vane, and diaphragm compressors are also viable options, particularly for very light gases at low power and high compression ratio.

The oil absorption column and regenerator suggested earlier for CH₄ separations 42 could also perform the main-process separations handled by cooled condensers 36, 37, 38, and 39 in FIG. 3. This could allow sufficient removal of the CO₂ without the boost compressor 35 and subsequent syngas expander 41, though energy-intensive re-compression of the CO₂, CO, and H₂ flashed from the oil is then required and other separations are more complex. The flash-gas from the oil regenerator would contain mostly CO₂ and CO along with the light HCs and a little H₂. If cryogenic separation is not used, it still may be necessary for the recycled syngas to be dehydrated to a low dew point. An absorption column using triethylene glycol (TEG, n.b.p.=551 K), which is commonly used to dehydrate various gas streams, is probably the best option. It may also be the best option for other gas dehydrating processes in the plant, especially if excess cooling capacity is not available (as might be the case when LOX is being produced).

There will for quite some time be an adequate market for the waste O₂, in which case it would probably be dried and liquefied by conventional processes (rather than being expanded in a heat engine 94). It is also possible that it would be piped at high pressure to another user, such as a methane-based GTL plant (where it could be used in POX). Making low-cost, high-purity oxygen widely available will likely lead to a dramatic reduction in the practice of gas flaring from oil fields, as it will then become practical to build much smaller methane-based GTL plants. The waste oxygen may also be useful in coal-fired power plants, as using oxygen rather than air simplifies CO₂ separation from the exhaust.

The focus herein has been on starting with clean hydrogen from electrolysis of high-pressure hot-water using wind energy because that is currently the most competitive source of renewable hydrogen. With future developments the cost of electrolysis-quality electrical energy on wind farms could be about 15% less than the cost of grid-quality wind energy, giving yet a further advantage to RFTS. There will be other alternatives for clean, renewable hydrogen in the future, as previously noted, some of which offer the advantage of low variability. The DORC, as described in a separate application, is likely to further improve the competitiveness of CSP, especially in areas where geothermal is also viable. Solar photovoltaic, perhaps using concentrators, again electrolyzing hot water, may become competitive in many places in the world before too long.

More advanced methods of electrolyzing water, such as proton exchange membranes also show promise, perhaps at temperatures as low as 340 K. Thermo-chemical dissociation of both water and CO₂ using concentrated solar at 1800-2500 K is being advocated by some, though it seems that more efficient routes to conversion of CSP to either electricity or hydrogen are more mature and likely to continue to be much more competitive. It is possible that H₂ and CO could eventually be produced simultaneously in high-temperature electrolysis of a steam/CO₂ mixture, as disclosed by Stoots et al in US Pub 2008002338. However, competitive results here, as in steam electrolysis, appear unlikely for the next 20 years, as known ceramic electrolytes are extremely expensive and fragile.

Conventional nuclear fission can hardly be called renewable because of the rate at which this resource is being consumed and because of the amount of CO₂ that could be released in the mining and processing of the low-grade, hard ores that will remain after 2018. However, advanced breeder reactors, if they could be shown to be sufficiently safe, could provide the needed source of hydrogen for several centuries. Some continue to believe that controlled thermonuclear fusion has potential, though realistic evaluations indicate that is almost as impractical as space solar power.

Some major industrial processes (most notably steel refining) currently produce enormous amounts of waste CO that have not often been well utilized, though biological processes are being developed for conversion of CO to ethanol. A more efficient use of waste CO would be in a process similar to that disclosed herein. The waste CO may be combined directly with renewable H₂ for conversion to hydrocarbons and alcohols in an FTS reactor. While this eliminates the need for the initial RWGS reactor, the RWGS reactor is still important with HT-FTS processes, as the HT-FTS processes generate considerable amounts of CO₂—via the WGS—which needs to be recycled through an RWGS reactor for reduction to CO.

There are many other chemical synthesis processes that utilize large quantities of H₂ and/or CO or CO₂. For example, oxosynthesis, also known as hydroformylation, involves the reaction of CO and H₂ with olefinic hydrocarbons to form an isomeric mixture of normal- and iso-aldehydes. The basic oxosynthesis reaction is highly exothermic, and it proceeds readily in the presence of homogeneous metal carbonyl catalysts. A renewable oxosynthesis plant could be built near an RFTS plant and use the renewable olefins from the RFTS plant along with renewable H₂ and CO to produce the desired valuable products, such as detergent-range (C₁₁-C₁₄) alcohols. Carbonylation of olefins with CO and a nucleophilic reaction partner with a labile H atom results in the formation of carboxylic acids or their derivatives, such as esters, thioesters, amides, and anhydrides.

As was seen in eq. [9], methanol can be made directly from CO₂ and H₂. However, CO-to-methanol conversion, the reverse of eq. [13], has been essential in commercial methanol production, where the ratio of CO/CO₂ in the feed gas has usually been greater than 5 and probably always greater than unity. Water from the reaction of eq. [9] apparently inhibits the production of methanol with common catalysts, though some believe this problem can be solved. It may be possible to produce various hydrocarbons from CO and water using a method similar to that disclosed by deVries in U.S. Pat. No. 5,714,657. Clearly, many processes could utilize a Renewable CO Production (RCOP) process, similar to that shown in either FIG. 4 or FIG. 5.

Renewable ammonia can be produced from renewable H₂ and nitrogen (separated from air). Eventually it may also make economic sense to deliberately produce methane from wind hydrogen and waste CO₂, which can be done with essentially 100% yield at over 670 K.

Some of the above processes would not need the RCOP process but would still benefit from VHP electrolysis of water. With renewable energy, variable-nozzle turbines or turbine switching in the above processes will permit improvements in cost effectiveness in the handling of the variable-rate H₂, O₂, CO₂, and N₂.

There are many obvious variations on an RFTS plant, even for production of mostly mid-alcohols, that were not mentioned in the various sections above. For example, the gaseous recycled CO₂ 49 could well contain large amounts of H₂, CO, and CH₄ (and thus change virtually all of the mixtures in predictable ways) in a design that permits cost reductions in some of the secondary separations. Better methods for separation of CH₄ from the high-pressure syngases, both new and recycled, and better methods for CO separation will undoubtedly be developed in the future. These separations, the HT-FTS catalysts, and the electrolyzer may be the most fruitful areas for improvements in the example plant outlined in FIG. 3.

Conclusions. The large uncertainty in the WGS activity in the HT-FTS reactor (which depends on catalysts developments and conditions) has little effect on overall system performance when the separations and recycling are efficiently handled. The design shown could handle considerably more H₂ and CO₂ production in the FTS reactors than assumed without difficulties.

Since approximately 99% of the products will be either sold, used, upgraded, or efficiently recycled, any likely changes in the FTS catalyst selectivity and operating conditions, resulting even in major changes in the product mix, will have only minor effects on the overall plant efficiency.

The key innovations include: (1) improving electrolysis efficiency by operating at higher pressure without losing the mechanical and thermal energy this puts into the gas streams; (2) dramatically improving efficiency of handling low-conversion FTS reactions by utilizing high-pressure cryogenic separations of the gases in a closed loop; (3) dramatically improving efficiency of low- or mid-temperature RWGS by either a recycle or a multi-stage process with optimized H₂O and CO separation processes; (4) dramatically improving cost-effectiveness of gas-to-gas recuperation, perhaps largely by methods disclosed in the above-mentioned co-pending application No. 61/034,148; and (5) utilizing more cost-effective reactor designs.

The high-value products will be mostly mid-alcohols, propylene, butenes, methanol, gasoline, jet fuel, diesel, and high-grade lubricant base stocks, with some ethylene, acetone, high alcohols, and other hydrocarbons and oxygenates. The amount of separations and upgrading that would be carried out at the local WindFuels plant would depend on the size of the plant. The product balance and the amount of recycling can change in response to the markets. The primary objective is to profitably convert over 90% of the carbon in the source CO₂ (or CO) into valuable liquid products. All products but methane are easily liquefiable for simplified distribution and storage, and a vast pipeline network is available for methane, though its sale may not be profitable before 2020. Some of the liquid hydrocarbon streams would require further refinement at a regional plant specifically designed for that purpose.

The near-term net plant HHV efficiency (relative to the renewable electrical input energy) is expected to be between 55% and 61%, depending on various capital investment trade offs and market conditions. This net efficiency could be slightly higher if low-grade waste heat is utilized in local steam heating, or it could be slightly lower if some lower value products are not counted in the products sum. Mid-term net efficiency should be 4-6% higher. Far more importantly, the innovations presented herein allow profitable production of many carbon-neutral fuels and petrochemicals at current [2008] market prices with only a moderate income stream from oxygen sales. Even with a very weak oxygen market, many petrochemicals could be produced profitably from wind and waste CO₂ in the markets likely by 2011.

Although this invention has been described herein with reference to specific embodiments, it will be recognized that changes and modifications may be made without departing from the spirit of the present invention. All such modifications and changes are intended to be included within the scope of the following claims.

1. A recycle Renewable CO Production (RCOP) method for producing carbon monoxide from electrical energy, water, and recovered CO₂, said method further characterized as including
using electrical energy to produce pressurized source hydrogen and source oxygen from water using an electrolyzer,
utilizing recovered CO₂ from effluent CO₂, chemical processes, natural gas, bodies of water, or the atmosphere,
utilizing an RWGS-gas recuperator for preheating reverse water gas shift (RWGS) reactants in preparation for an RWGS reaction in a catalytic reactor while cooling RWGS products in preparation for water condensation,
further heating the preheated RWGS reactants in a heat exchanger,
utilizing an RWGS reactor for at least partial conversion of CO₂ and H₂ in RWGS reactants to CO and H₂O in RWGS reactor products at mean RWGS operating temperature above 550 K and at RWGS operating pressure above 0.2 MPa,
condensing most of the water from the cooled RWGS products,
separating a major fraction of the CO from the RWGS products, and
recycling a major fraction of the un-reacted H₂, CO₂, un-separated CO, and un-separated water through said RWGS-gas recuperator and subsequent components.

2. The recycle RCOP method of 1 in which said electrolyzer is further characterized as capable of generating hydrogen and oxygen from water at pressure between 0.3 MPa and 70 MPa and temperature between 340 K and 520 K.

3. The recycle RCOP method of 1 further characterized as including a gas heat engine comprising an electrical generator and an expansion turbine driven by a stream of pressurized expander gas, said expander gas further characterized as having molar fraction of H₂O less than 50% but greater than 0.5% and the balance of said gas comprised substantially of a single molecular species.

4. The recycle RCOP method of 1 in which said RWGS-gas recuperator is further characterized as having thermal effectiveness greater than 80% and including tens of thousands of parallel gas flow passages of hydraulic diameter less than 8 mm.

5. The recycle RCOP method of 1 in which said RWGS reactor products have molar fractions of CO and H₂O less than 0.2 and 0.15 respectively.

6. The recycle RCOP method of 1 further characterized in that the maximum sum of the H₂ and CO₂ partial pressures within a primary recycle loop is less than twice the minimum sum of the H₂ and CO₂ partial pressures within the primary recycle loop.

7. The recycle RCOP method of 1 further characterized as utilizing an absorption column with CuCl and AlCl₃ in an organic solvent for separating CO from said RWGS products.

8. (canceled)

9. The recycle RCOP method of 1 in which said RWGS reactor system includes catalysts from the set comprising copper on silica, copper on γ-alumina, and Fe₃O₄/Cr₂O₃.

10. The recycle RCOP method of 1 further characterized as including a cryogenic oxygen expander turbine and a cryogenic oxygen heat exchanger for the production of liquid oxygen.

11. A multi-stage Renewable CO Production (RCOP) method for producing carbon monoxide from electrical energy, water, and recovered CO₂, said method further characterized as including
using electrical energy to produce pressurized source hydrogen and source oxygen from water using an electrolyzer,
utilizing recovered CO₂ from effluent CO₂, chemical processes, natural gas, bodies of water, or the atmosphere,
sending primary reactants CO₂ and H₂, sequentially through a plurality of reverse water gas shift (RWGS) stages, wherein reactants also include a minimum molar composition of 21% CO,
each said RWGS stage comprising
an RWGS-gas recuperator for preheating reactants in preparation for an RWGS reaction while cooling RWGS products in preparation for water condensation,
a heat exchanger for further heating the preheated RWGS reactants,
an RWGS reactor for at least partial conversion of said RWGS reactants to CO and H₂O in RWGS reactor products at mean RWGS operating temperature above 550 K and at RWGS operating pressure above 0.2 MPa,
a condenser for separating most of the water from the cooled RWGS products.

12. The multi-stage RCOP method of 11 in which said electrolyzer is further characterized as capable of generating hydrogen and oxygen from water at pressure between 0.3 MPa and 70 MPa and temperature between 340 K and 520 K.

13. The multi-stage RCOP method of 11 further characterized as including a gas heat engine comprising an electrical generator and an expansion turbine driven by a stream of pressurized expander gas, said expander gas further characterized as having molar fraction of H₂O less than 50% but greater than 0.5% and the balance of said gas comprised substantially of a single molecular species.

14. (canceled)

15. The multi-stage RCOP method of 11 in which said RWGS reactor products have molar composition of H₂O less than 10%.

16. (canceled)

17. The multi-stage RCOP method of 11 in which said RWGS reactor system includes catalysts from the set comprising copper on silica, copper on γ-alumina, and Fe₃O₄/Cr₂O₃.

18. The multi-stage RCOP method of 11 further characterized as including a cryogenic oxygen expander turbine and a cryogenic oxygen heat exchanger for the production of liquid oxygen.

19. A Renewable Fischer Tropsch Synthesis (RFTS) process for producing hydrocarbons and oxygenates from electrical energy, water, and recovered CO₂, said process further characterized as including
using electrical energy to produce compressed source hydrogen and source oxygen from preheated water using an electrolyzer,
utilizing recovered CO₂ from effluent CO₂, chemical processes, natural gas, bodies of water, or the atmosphere,
utilizing a reverse water gas shift (RWGS) reactor system for at least partial conversion of CO₂ and H₂ in RWGS reactants to CO and H₂O in RWGS products at mean RWGS operating temperature above 550 K and at RWGS operating pressure above 0.12 MPa,
utilizing an RWGS-gas recuperator for cooling RWGS products in preparation for water condensation while preheating RWGS reactants in preparation for an RWGS reaction in a catalytic reactor to produce new syngas,
using an exothermic FTS reactor for the catalytic production of FTS products, including hydrocarbons, CO, H₂, CO₂, and water, at a desired mass flow rate, from feed syngas that includes H₂, CO, and CO₂, said FTS reactor operating at FTS mean temperature greater than 450 K,
using fractional separation means to produce multiple streams of FTS liquid products at sequentially lower temperatures, and
including means for recycling a major fraction of the CO and H₂ from the FTS products back into the FTS reactor in a stream herein denoted final recycled syngas.

20. The RFTS process of 19 in which a plurality of said multiple streams is condensed at a pressure greater than one half of the mean gas total pressure in said FTS reactor.

21. The RFTS process of 19 further characterized as including a gas heat engine comprising an electrical generator and an expansion turbine driven by a stream of pressurized expander gas heated by thermal communication with said FTS reactor to a temperature greater than said FTS temperature minus 100 K, said expander gas further characterized as having molar fraction of H₂O less than 50% but greater than 0.5%.

22. The RFTS process of 19 further characterized as including heat exchanger control means for maintaining the temperatures of said FTS reactor, RWGS reactors, and said separation means near predetermined optimal temperatures which are dependent on the mass flow rate of said source hydrogen while said mass flow rate changes over a range greater than a factor of two from minimum to maximum flow rate.

23. The process of 19 in which said electrolyzer is further characterized as capable of generating hydrogen and oxygen from water at pressure in excess of 2 MPa and at temperature greater than 400 K.

24. The RFTS process of 19 further characterized as including a final syngas heat exchanger for transfer of heat from FTS products to the final recycled syngas.

25. The RFTS process of 19 further characterized as including a new-syngas compressor for increasing the pressure of said new syngas by a pressure ratio greater than 1.3, said compressor output herein denoted compressed new syngas,

26. The RFTS process of 19 in which said fractional separation means is further characterized as including recovery of thermal energy from said FTS products at multiple temperatures.

27. The RFTS process of 19 further characterized as including a cryogenic condenser and a recycle-loop boost compressor for increasing the pressure of said FTS products in said cryogenic condenser to a pressure greater than 1.1 times that in said FTS reactor.

28. The process of 19 further characterized in that said RWGS operating pressure is between 0.2 MPa and 5 MPa.

29. (canceled)

30. The RFTS process of 19 further characterized as having H₂/CO molar ratio in the final recycled syngas less than 1.4.

31. The RFTS process of 19 further characterized as having recycled syngas mass flow greater than said new syngas mass flow.

32. (canceled)

33. The RFTS process of 19 further characterized as including an O₂ refrigerator comprising said compressed source O₂, heat transfer means in communication with the environment for cooling said source O₂ to RT-O₂ having temperature below 340 K, and a multi-stage expansion turbine for expanding said RT-O₂ to atmospheric pressure through a heat exchanger in thermal communication with separation means.

34. The RFTS process of 19 further characterized as including means for separating a major fraction of the CO₂ from said RWGS products and means for recycling this RWGS-CO₂ through said RWGS reactor.

35. The RFTS process of 19 further characterized as including means for separating a substantial fraction of the CO₂ from the products of said FTS reactor into a stream herein denoted FTS-CO₂, and means for recycling this FTS-CO₂ through said RWGS reactor.

36. The RFTS process of 19 in which said RWGS reactor system includes multiple, series-connected, heat-react-condense stages.

37. The RFTS process of 19 in which said RWGS reactor system includes means for CO-separation from said RWGS products and recycling of un-reacted H₂ and CO₂ through said RWGS recuperator.

38. The RFTS process of 19 further characterized as including a chilled oil absorption column for separating inert gases from recycled syngas, inert gases herein defined as including methane, argon, and nitrogen.

39. (canceled)

40. The process of 19 further characterized as utilizing a portion of said source oxygen and a portion of said FTS products to produce syngas using exothermic catalytic partial oxidation (CPOX).

41. The RFTS process of 19 further characterized as utilizing a fluid of normal boiling point greater than 450 K to transfer heat into said RWGS reactor, said fluid selected from the set comprised of molten alloys, molten salts, and organics.

42. The process of 19 further characterized in that a portion of said feed syngas includes waste CO from an industrial process.

43. The process of 19 further characterized in that said RWGS operating temperature is below 900 K.

44. The RFTS process of 19 in which dissolved CO₂ is flashed to CO₂ gas from at least one of said multiple liquid streams and said liquid stream is further used for partial cooling of a condenser.

45. The RFTS process of 19 further characterized as including means for storing said source hydrogen gas in quantities greater than 300 kg of H₂ at a plurality of temperatures and pressures.

46. The RFTS process of 19 further characterized as including means for preventing venting of more than 30% of the subset of FTS hydrocarbon products that have boiling points below 320 K at atmospheric pressure.

47. The RFTS process of 21 in which said expander gas is further characterized as having molar fraction greater than 50% of a gas selected from the set comprising CO₂, H₂, and O₂.

48. (canceled)

49. (canceled)

50. The RFTS process of 27 further characterized as including a recuperator for cooling a warmer portion of a recycled syngas stream against a cooler portion of a recycled syngas stream, said recuperator further characterized as utilizing flow passages of hydraulic diameter less than 4 mm.

51. The RFTS process of 27 further characterized in that the total pressure in said cryogenic condenser is greater than 8 MPa.

52. The RFTS process of 32 further characterized in that the major constituent of one of said multiple liquid streams is selected from the set comprising ethanol, propanols, butanols, and alkanes having from 6 to 10 carbon atoms.

53. The RFTS process of 33 further characterized as utilizing means for storing oxygen in amounts greater than 2000 kg of O₂.

54. The RFTS process of 36 in which said RWGS reactor system includes Fe₃O₄/Cr₂O₃ catalysts.

55. The RFTS process of 37 in which said means for CO-separation is further characterized as including an absorption column containing a solution of CuAlCl₄ in an aromatic solvent.

56. The RFTS process of 37 in which said RWGS reactor includes catalysts from the set comprising copper on silica and copper on γ-alumina.

57. The RFTS process of 40 in which the CPOX reaction occurs at a temperature greater than said RWGS operating temperature and some of the heat from the CPOX reaction is transferred to the RWGS reaction.

58. (canceled)

59. (canceled)

60. A multi-tubular, fixed-bed reverse water gas shift (RWGS) reactor for the catalytic production of CO and H₂O from CO₂ and H₂, said RWGS reactor further characterized as utilizing a heating liquid having normal boiling point greater than 550 K selected from the set comprised of molten alloys, molten salts, and organic liquids.
